(19) United States
(12) Patent Application Publication
(10) Pub. No.: US 2020/0058376 A1
(43) Pub. Date: Feb. 20, 2020

(54) BIOREACHABLE PREDICTION TOOL FOR PREDICTING PROPERTIES OF BIOREACHABLE MOLECULES AND RELATED MATERIALS

(71) Applicant: Zymergen Inc., Emeryville, CA (US)

(72) Inventors: Erik Jedediah Dean, Lafayette, CA (US); Vanessa Blue Oklejas, Piedmont, CA (US); Alexander Glennon Shearer, San Francisco, CA (US); Vytas SunSpiral, Oakland, CA (US); Michelle L. Wynn, Alameda, CA (US); Lucas Andrew Zulauf, Emeryville, CA (US)

(73) Assignee: Zymergen Inc., Emeryville, CA (US)

(21) Appl. No.: 16/542,157

(22) Filed: Aug. 15, 2019

Related U.S. Application Data
(60) Provisional application No. 62/720,839, filed on Aug. 21, 2018.
(60) Provisional application No. 62/764,861, filed on Aug. 15, 2018.

(51) Int. Cl. G16B 40/00; G06N 7/00; G06N 20/00; G16B 5/20

(52) U.S. Cl. CPC G16B 40/00; G16B 5/20; G06N 20/00; G06N 7/005

(57) ABSTRACT

Systems, methods and computer-readable media are provided to predict properties of a material that is related to a bioreachable molecule by generating a chemical model of the material based on physicochemical properties and predicting properties of the material based at least in part upon the chemical model and correlative modeling. The material may comprise in its chemical structure one or more instances of the bioreachable molecule. The material may comprise in its chemical structure the bioreachable molecule or at least one semi-synthetic molecule derived from the bioreachable molecule, or a combination thereof.

### CROSS-REFERENCE TO RELATED APPLICATIONS

This application claims the benefit of U.S. Provisional Application No. 62/764,861, filed Aug. 15, 2018, and U.S. Provisional Application No. 62/720,839, filed Aug. 21, 2018, both of which are incorporated by reference in their entirety herein.

This application is related to P.C.T. Application No. PCT/US2018/018234, filed on Feb. 14, 2018 (the “BPT PCT application”), which claims the benefit of U.S. Provisional Application No. 62/459,558, filed Feb. 15, 2017, both of which are incorporated by reference in their entirety herein.

### FIELD

The disclosure relates generally to molecular and materials innovation, and in particular to determining bioreachable molecules and related molecules and materials that possess desired properties.

### BACKGROUND

Biologists, chemists, material scientists, and others in related disciplines employ bioengineering to produce desired molecules with desired phenotypic characteristics from cells by, for example, modifying the cell's genome. Such cells may themselves be unicellular organisms (e.g., bacteria) or components of or multicellular host organisms, or may be mutated variants of cells found in nature. However, there is a limit to which molecules can be produced as part of the biomass in a cell. Generally, one is faced with the problem of determining the largest possible pool of bioreachable molecules that may be generated through genetic modification without requiring extensive manual intervention. This problem was addressed in the BPT PCT application.

To date, progress has been made in assessing a subset of the molecules predicted as bioreachable using embodiments of the disclosure described herein and in the BPT PCT application. Researchers may order research samples of these molecules. These molecules may be too expensive to use commercially with current synthesis processes, but they allow researchers to explore application use cases and develop titer tests.

Currently, in order to decide which molecules to order and explore, chemists are often limited to inspecting the structural elements of the molecules and using their intuition to estimate properties of these molecules. Specialized databases have been created based on some structural features in order to help chemists search for molecules with those features. However, this approach only provides a rough, and likely incomplete, guess based on existing intuition as to how the molecules will behave. Experience has already shown that bioreachable molecules predicted by embodiments of the disclosure described herein and in the BPT PCT application can have surprising behaviors that existing ad hoc approaches do not capture or predict. Researchers have been practically limited to the molecules for which samples can be purchased in order to physically verify expectations about the molecule.

### SUMMARY OF THE DISCLOSURE

To unlock the potential of putative bioreachable materials for which samples cannot be easily purchased, embodiments of the disclosure compute basic chemical properties of these molecules to provide a starting point to reason about their use in diverse applications. Embodiments of the disclosure also predict properties of materials comprising one or more actual or likely bioreachable molecules. These computed basic properties of the likely bioreachable molecules and materials, which are calculated based on a minimum of a priori information, serve as a platform from which an experienced materials scientist can make reasonably educated guesses as to how to use these new building block molecules in new, advanced materials.

Embodiments of the disclosure employ computational molecular modeling (CMM), which stems from the inventors' vision of producing novel bioreachable molecules that have never been synthesized before, and that have unique properties (e.g., chirality, multiple functional groups, which may serve as “handles” for subsequent chemical transformations) not commonly found in building block chemicals used in traditional synthetic chemistry methods.

Attaining this objective presents some key challenges:

We do not know the properties of these molecules, and they have not been studied, so we do not know what applications they may be good for.

Because they have unique new aspects, it is not clear if one can reasonably extrapolate properties based upon other “traditionally synthesized” molecules.

It is expensive and difficult to make many of these new molecules for the first time, so a researcher would want to know if there is a good application for a molecule before generating a first physical sample of the molecule

To overcome these challenges, embodiments of the disclosure provide systems, methods and computer-readable media for predicting at least one property of a first molecule of one or more putative bioreachable molecules. Such embodiments generate a chemical model of the first molecule based on physicochemical properties; and predict the at least one property of the first molecule based at least in part upon the chemical model and empirical data concerning the first molecule, wherein the empirical data does not include data concerning the at least one property. The first molecule may then be physically obtained.

Predicting the at least one property of the first molecule may employ statistical modeling or machine learning. At least one of the one or more putative bioreachable molecules may be determined by: obtaining, using at least one processor, a starting metabolite set specifying starting metabolites for a host organism; obtaining, using at least one processor, a starting reaction set specifying reactions; using at least one processor, including in a filtered reaction set one or more reactions from the starting reaction set; and in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

Embodiments of the disclosure provide systems, methods and computer-readable media for identifying a putative bioreachable molecule having a desired property. Such embodiments (1) receive a query of a database, the query indicating a desired property, wherein (a) the database stores associations between one or putative bioreachable molecules and one or more predicted properties of the one or more putative bioreachable molecules, and (b) the one or more predicted properties include the desired property; and (2) return data representing a first putative bioreachable molecule of the one or more putative bioreachable molecules based at least in part upon association of the first putative bioreachable molecule with the desired property.

The one or more predicted properties may be based at least in part upon chemical modeling of the one or more putative bioreachable molecules. The one or more predicted properties may be based at least in part upon empirical data concerning the one or more putative bioreachable molecules. The one or more predicted properties may be based at least in part upon statistical modeling or machine learning.

Embodiments of the disclosure provide systems, methods and computer-readable media for predicting at least one property of a material related to a first molecule of one or more putative bioreachable molecules. Such embodiments generate a chemical model of the material based on physicochemical properties; and predict the at least one property of the material based at least in part upon the chemical model and correlative modeling.

Predicting the at least one property of the material may be based at least in part upon predictive modeling of the first molecule. The material may comprise at least the first molecule. The material may comprise at least two instances of the first molecule in its structure. The material may comprise in its chemical structure at least the first molecule or at least one semi-synthetic molecule derived from the first molecule, or a combination thereof.

Embodiments of the disclosure provide systems, methods and computer-readable media for identifying a material having a desired material property, wherein the material is related to one or more putative bioreachable molecules. Such embodiments receive a query of a database, the query indicating a desired material property, wherein the database stores associations between one or more materials and one or more predicted material properties of the one or more materials, and the one or more predicted material properties include the desired material property, the one or more predicted material properties are based at least in part upon chemical modeling of the one or more putative bioreachable molecules; and return data representing a first material of the one or more materials based at least in part upon association of the first material with the desired material property. The associations may be based at least in part upon statistical modeling or machine learning.

The first material may comprise in its chemical structure at least one bioreachable molecule of the one or more putative bioreachable molecules or at least one semi-synthetic molecule, or a combination thereof.

Embodiments of the disclosure provide systems, methods and computer-readable media for identifying one or more putative bioreachable molecules related to a desired material property. Such embodiments receive a query of a database, the query indicating a desired material property, wherein the database stores associations between one or more putative bioreachable molecules and material properties of one or more materials; and return data representing one or more putative base bioreachable molecules of the one or more putative bioreachable molecules, wherein the one or more putative base bioreachable molecules are related to at least one material, of the one or more materials, that has the desired material property. The returned data may include data representing at least one reaction pathway between the one or more putative base bioreachable molecules and the at least one related material. The at least one related material may be obtained via chemical transformation from at least one of the one or more putative base bioreachable molecules.

At least one related material may include within its structure the one or more putative base bioreachable molecules. The at least one related material may be a polymer and each putative base bioreachable molecule of the one or more putative base bioreachable molecules may be a monomer. The at least one related material may comprise in its chemical structure at least one base bioreachable molecule of the one or more putative base bioreachable molecules or at least one semi-synthetic molecule, or a combination thereof. The association between the one or more putative base bioreachable molecules and the desired material property may be based at least in part upon predictive modeling, which may employ statistical modeling or machine learning.

Embodiments of the disclosure provide systems, methods and computer-readable media for predicting properties of molecules derived from putative bioreachable molecules. Such embodiments transform in silico a first putative bioreachable molecule of one or more putative bioreachable molecules to produce a second molecule; and predict at least one property of the second molecule. Predicting the at least one property may be based at least in part upon chemical modeling of the first putative bioreachable molecule. Predicting the at least one property may be based at least in part upon machine learning. Transforming may involve chemically transformation, and may require at most 2 or 3 reaction steps.

### BRIEF DESCRIPTION OF THE DRAWINGS

FIG. 1 illustrates a system for implementing a bioreachable prediction tool according to embodiments of the disclosure.

FIG. 2 is a flow diagram illustrating operation of a bioreachable prediction tool according to embodiments of the disclosure.

FIG. 3 illustrates pseudocode for implementing strict and relaxed enzyme sequence searches according to embodiments of the disclosure.

FIG. 4 illustrates an example of a report that may be generated by the bioreachable prediction tool of embodiments of the disclosure.

FIG. 5 illustrates a hypothetical example of a report of reaction pedigree tracking that may be generated by the bioreachable prediction tool of embodiments of the disclosure.

FIG. 6 illustrates a cloud computing environment according to embodiments of the disclosure.

FIG. 7 illustrates an example of a computer system that may be used to execute instructions stored in a non-transitory computer readable medium (e.g., memory) in accordance with embodiments of the disclosure.

FIG. 8 illustrates an example of a single pathway of the type that may be generated by the biroeachable prediction tool of embodiments of the disclosure. In this example, the molecule tyramine was predicted to be reachable by addition of a single enzymatic step to a host organism. This pathway has been reduced to practice and engineered into host organisms to produce tyramine. This pathway's evaluation score is included in the reaction diagram.

FIG. 9 illustrates an example of two distinct pathways of the type that may be generated by the bioreachable prediction tool of embodiments of the disclosure. In this example, both pathways were identified by the bioreachable prediction tool as being able to generate the bioreachable molecule (S)-2,3,4,5-tetrahydrodipicolinate (THDP). The two pathways differ by their use of reducing equivalent types (NADH versus NADPH). One of these pathways has been reduced to practice and engineered into host organisms to produce THDP. Each pathway's evaluation score is included in the reaction diagram.

FIG. 10 illustrates an example of a more complex multi-pathway prediction of the type that may be generated by the bioreachable prediction tool of embodiments of the disclosure. Each pathway's evaluation score is included in the reaction diagram.

FIGS. 11A and 11B together illustrates an example of a scoring breakdown that may be generated by the bioreachable prediction tool of embodiments of the disclosure. (FIG. 11B appends to the bottom of FIG. 11A.) In this case, the evaluation data shown was generated during the process of predicting pathways to the molecule (S)-2,3,4,5-tetrahydrodipicolinate (THDP).

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

The inventors have recognized that conventional methods for predicting viable target molecules suffer from the following obstacles:

1) Lack of biological parts. This is the single biggest cause of false positive predictions about chemicals that can be biologically generated. Some conventional methods employ existing reaction databases to step through all known metabolic reactions from a feedstock like glucose, and assume all paths can be engineered. However, many reactions do not correspond to a genetic part that can be engineered into a host organism. Typically, reactions are catalyzed by enzymes. Reactions in existing databases may be well-characterized according to their catalyzing enzymes, but many of those enzymes have not had their amino acids sequenced, meaning that there is no established correlation between the enzyme and an associated gene sequence. Without a gene sequence, a host genome cannot be modified to produce the required enzyme. In fact, approximately 25-50% of well-characterized enzymatic reactions have no known associated gene sequence, and thus those enzymes are not useful as biological parts for engineering purposes. The percentage of gene-absent reactions in overall biological databases is likely even higher, since these databases include many reactions that are not well-characterized. The inventors note that, in some cases, catalysts other than enzymes, such as enzyme-nanoparticle conjugates, may be employed. See, e.g., Vertgel A A, et al., Enzyme-nanoparticle conjugates for biomedical applications, Methods Mol. Bio. 2011; 679: 165-82; Johnson P A, et al., Enzyme nanoparticle fabrication: magnetic nanoparticle synthesis and enzyme immobilization, Methods Mol. Biol. 2011; 679: 183-91, all of which are incorporated by reference in their entirety herein. In those cases, parts required to engineer those catalysts into a host organism may or may not be known.

2) Incorrect pathway tracing. Many attempted solutions attempt to trace pathways arbitrarily between molecules. This can lead to a failure to properly track the creation of the target molecule's carbon skeleton. To cite a common example, a path might be traced from glutamine into a reaction that generates a target molecule, and then glutamine would be cited as a part of the path to creating that target molecule. However, in most cases, glutamine is donating a nitrogen group and no carbon, so this tracing is misleading and does not indicate that the target molecule can be made (other errors include tracing connections though other ubiquitous molecules such as ATP or inorganic molecules such as water). These types of pathway tracing errors also lead to an unusably large number of predicted pathways (as if a mapping application allowed all possible street routes through San Francisco, instead of the two to three most direct and useful paths).

3) Assuming bidirectional reactions. Another significant source of errors is failure to account for the thermodynamics/direction of reactions. Thermodynamics dictates that some reactions can only run in one direction. A reaction that only degrades molecule A to molecule B, however, is often predicted by conventional means to run in either direction, so it would be incorrectly predicted that molecule A can be synthesized from B. As a particular example, some bacteria breakdown halogenated compounds such as organochlorides, but cannot run in reverse to create halogenated compounds. Because many biological reactions are dramatically favored to run in only one direction, failure to account for reaction directionality would also create a false positive prediction.

4) Other errors. Not every host can be engineered to produce every target molecule, or be engineered to produce every target molecule with the same set of modifications or likelihood of success, because not all hosts maintain the same set of metabolic pathways.

The bioreachable prediction tool (BPT) of embodiments of the disclosure overcomes the limitations of conventional methods. The BPT of embodiments of the disclosure may describe, in a target-agnostic fashion, every chemical that likely can be biologically generated given a set of starting constraints (e.g. particular host organism, number of reaction steps, whether only reactions with gene-sequenced enzymes allowed). This creates a “bioreachable list,” a list of viable target chemicals. These target chemicals and their associated structures can be provided to professional chemists, who can review the chemical utility of the molecules without having to consider the biology required to create them. After particular bioreachable target chemicals are selected, their formulas and reaction pathways may be provided to a gene manufacturing system to modify the gene sequence of the host organism to produce the selected target molecules.

Embodiments of the disclosure provide a bioreachable prediction tool for predicting viable target molecules in a manner that overcomes the disadvantages of conventional techniques. In particular, the bioreachable prediction tool of the present disclosure predicts viable target molecules that are specific to a specified host organism.

The bioreachable prediction tool of embodiments of the disclosure obtains a starting metabolite set specifying starting metabolites for the host cell. In embodiments, the starting metabolite set specifies core metabolites, the core metabolites including metabolites indicated by at least one database as produced by an un-engineered host under specified conditions. In embodiments, the host has not been subjected to genetic modification.

In embodiments, the bioreachable prediction tool obtains a starting reaction set specifying reactions. In embodiments, the tool includes in a filtered reaction set one or more reactions from the starting reaction set that are indicated in at least one database as catalyzed by one or more corresponding catalysts, e.g., enzymes, that are themselves indicated as likely available to catalyze the one or more reactions that may take place in the host cell.

A catalyst is likely “available to catalyze” a reaction in a host cell if the bioreachable prediction tool determines information from, e.g., public or proprietary databases, indicating that the catalyst may be introduced into the host either by engineering the catalyst into the host (e.g., by modifying the host genome, adding a plasmid) or via uptake of the catalyst from the growth medium in which the host is grown.

More specifically, this disclosure refers to a part, such as a catalyst, as being “engineered” into a host cell when the genome of the host cell is modified (e.g., via insertion, deletion, replacement of genes, including insertion of a plasmid coded for production of the part) so that the host cell produces the catalyst (e.g., an enzyme protein). If, however, the part itself comprises genetic material (e.g. a nucleic acid sequence acting as an enzyme), the “engineering” of that part into the host cell refers to modifying the host genome to embody that part itself.

A part is likely “available to be engineered” into the host cell if the bioreachable prediction tool determines information indicating that the part can be engineered in the host. For example, according to embodiments, the tool would determine information indicating that an enzyme is likely available to be engineered into a host if the enzyme is found to be engineerable into the host, e.g., as indicated by annotation in a public or proprietary database accessed by the BPT tool. If there is evidence that at least one amino acid sequence is known (e.g., found in one of the above databases) to catalyze the reaction (in any host), then skilled artisans would be able to derive the corresponding genetic sequence used to code the amino acid sequence, and modify the host genome accordingly. If the likely available part is an enzyme, the tool can select a set of enzyme sequences predicted as highly likely to catalyze a reaction needed to make the molecule, where an enzyme sequence may be represented as a protein amino acid sequence or genetically as DNA or RNA, and may be native or heterologous. In this context and in the claims, “likely” means more probable than not, i.e., having a greater than 50% likelihood.

In each processing step of one or more processing steps leading to prediction of a bioreachable molecule, the bioreachable prediction tool processes, pursuant to the one or more reactions of the filtered reaction set, data representing the starting metabolites and metabolites generated in previous processing steps, to generate data representing one or more viable target molecules. The tool provides, as output, data representing the one or more viable target molecules.

In embodiments, the bioreachable prediction tool determines a degree of confidence as to whether a corresponding catalyst is available to catalyze the one or more reactions in the host cell, e.g., available to be engineered into the host cell to catalyze the one or more reactions. The degree of confidence may include, for example, at least a first degree of confidence or a second degree of confidence higher than the first degree of confidence. The tool may include, in the filtered reaction set, one or more reactions from the starting reaction set that are indicated in at least one database as catalyzed by one or more corresponding catalysts that are themselves determined to be available, with the second degree of confidence, to catalyze the one or more reactions in the host cell, e.g., determined to be available, with the second degree of confidence, for engineering into the host cell to catalyze the one or more reactions.

In embodiments of the disclosure, the bioreachable prediction tool generates an indication of the difficulty of producing one or more of the viable target molecules. The indication of difficulty may be based upon thermodynamic properties, reaction pathway length for the one or more viable target molecules, or a degree of confidence as to whether a catalyst is available to catalyze one or more corresponding reactions along one or more first reaction pathways to one or more of the viable target molecules.

In embodiments of the disclosure, after generating data representing one or more viable target molecules in a particular processing step and before the next processing step, the bioreachable prediction tool removes from the filtered reaction set any reactions associated with generating the data representing one or more viable target molecules in the particular processing step.

In embodiments, the tool generates a record of one or more reaction pathways (i.e., pedigrees) leading to each viable target molecule. In embodiments, generating a record comprises not including in the record reaction pathways from ubiquitous metabolites. In embodiments, the tool generates a record of the step in which data representing a viable target molecule is generated. In embodiments, the tool generates a record of the shortest reaction pathway from the starting metabolite set to each viable target molecule.

Instead of determining viable target molecules given a single host cell, it may be desired to identify one or more host cells in which to produce a given viable target molecule. For example, a customer may ask the user of the tool to determine the optimum host cell within a plurality of hosts in which to produce the target molecule. In embodiments, the bioreachable prediction tool is run for a plurality of host cells, and generates data representing one or more viable target molecules (bioreachable candidate molecules), according to any of the methods described herein, for each host cell of the plurality of host cells. In such embodiments, for a given viable target molecule, the tool determines at least one of the plurality of host cells that satisfies at least one criterion, such as a given predicted yield of the viable target molecule produced by a given host cell or a given number of processing steps predicted as necessary to produce the given viable target molecule in a given host cell. The tool provides, as output, data representing the host cells determined to satisfy the at least one criterion.

As described for embodiments above, the tool may generate a record, including, e.g, thermodynamic properties, of one or more reaction pathways (i.e., pedigrees) leading to each target molecule produced by each host cell. Based on the above embodiments running the tool for the plurality of host cells, the tool may store associations between host cells, target molecules, and pedigrees in a database as a library, which may include annotations specifying parameters such as yield, number of processing steps, availability of catalysts to catalyze reactions in the reaction pathways, etc.

In embodiments, if the tool has access to such a library, the tool need not be run to identify multiple host cells in which to produce a given viable target molecule. Instead, in such embodiments, the tool may use the pedigrees from the library, which may include annotation data concerning associations among the hosts, target molecules, and reactions. The tool may identify at least one target host cell from among the one or more host cells based at least in part upon evidence, from, e.g., public or proprietary databases or from the library, that all the catalysts predicted to catalyze reactions in at least one reaction pathway leading to production of the target molecule in the at least one target host cell are likely available to catalyze all such reactions. In embodiments, the tool may determine target hosts based upon the target hosts requiring less than a threshold number of reaction steps within the reaction pathways that are predicted as necessary to produce the target molecule.

Some reaction enzymes may not have a known associated amino acid sequence or genetic sequence (“orphan enzymes”). In such cases, the tool may instead bioprospect the orphan enzymes to predict their amino acid sequences, and, ultimately, their genetic sequences, so that the newly-sequenced enzymes may be engineered into the host cell to catalyze one or more reactions. The tool may include the reactions corresponding to the newly-sequenced enzymes as members of the filtered reaction data used for bioreachable molecule finding.

In embodiments, the bioreachable prediction tool provides to a “factory,” e.g, a gene manufacturing system, an indication of one or more genetic sequences associated with one or more reactions in a reaction pathway leading to a viable target molecule. In embodiments, the gene manufacturing system embodies the indicated genetic sequences into the genome of the host, to thereby produce an engineered genome for manufacture of the target molecule. In embodiments, the tool provides to the factory an indication of one or more catalysts for the factory to introduce the one or more catalysts into the growth medium of the host cell for production of the target molecule.

In embodiments, the bioreachable prediction tool includes, in the filtered reaction set, reactions from the starting reaction set based at least in part upon whether the one or more reactions are spontaneous, based at least in part upon their directionality, based at least in part upon whether the one or more reactions are transport reactions, or based at least in part upon whether the one or more reactions generate a halogen compound.

In embodiments of the disclosure, the bioreachable prediction tool obtains a starting metabolite set specifying starting metabolites for the host cell, and obtains a starting reaction set specifying reactions specific to the host. In embodiments of the disclosure, the bioreachable prediction tool includes in a filtered reaction set one or more reactions that are indicated as spontaneous in at least one database. In each processing step of one or more processing steps, the tool processes, pursuant to the one or more reactions of the filtered reaction set, data representing the starting metabolites and any metabolites generated in previous processing steps, to generate data representing one or more viable target molecules in each step. In embodiments, the tool provides, as output, data representing the one or more viable target molecules.

System Design

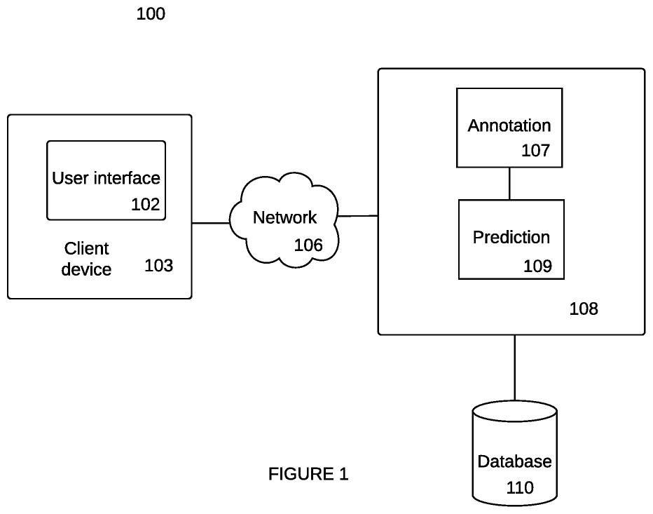

FIG. 1 illustrates a distributed system 100 of embodiments of the disclosure. A user interface 102 includes a client-side interface such as a text editor or a graphical user interface (GUI). The user interface 102 may reside at a client-side computing device 103, such as a laptop or desktop computer. The client-side computing device 103 is coupled to one or more servers 108 through a network 106, such as the Internet.

The server(s) 108 are coupled locally or remotely to one or more databases 110, which may include one or more corpora of molecule, reaction, and sequence data. The reaction data may represent the set of all known metabolic reactions. In embodiments, the reaction data is universal, i.e., not host-specific.

The molecule data includes data on metabolites-reactants involved in the reactions contained in the reaction data as either substrates or products. In embodiments, the data on metabolites includes data on host-specific metabolites, such as core metabolites, known in the art to be produced in particular host cells. In some embodiments, some core metabolites were determined to be produced by a particular host through empirical evidence gathered by the inventors. These host-specific metabolite sets were identified through various methods such as metabolomics analysis of the host cell or by identifying enzyme-coding genes that are essential under certain growth conditions, and inferring the presence of metabolites produced by the enzymes coded by those genes. The molecule data may be tagged with annotations representing many features, such as host cell, growth medium characteristics, and whether a molecule is a core metabolite, a precursor, ubiquitous, or inorganic.

The database(s) 110, e.g., UniProt, may also include data on whether a catalyst may be introduced into a host cell via uptake of the catalyst from a growth medium in which the host is grown.

The sequence data may include data for the reaction annotation engine 107 to annotate reactions in the reaction data set as to whether a reaction is likely known to correspond to sequences, e.g., enzyme or genetic sequences, for engineering the reaction into a host cell. For example, the sequence data may include data for annotating reactions in the reaction data as to whether a reaction is catalyzed by an enzyme for which the corresponding amino acid sequence is likely known. If so, then, through methods known in the art, a genetic sequence for coding the enzyme can be determined. In embodiments, for purposes of determining viable target molecules, the reaction annotation engine 107 does not need to know the sequence data itself, but rather only whether a sequence is likely known to exist for the catalyst. The reaction annotation engine 107, described below, may compile the sequence data from databases such as UniProt, which include sequence data for enzymes that catalyze reactions indicated as having associated coding sequences. The sequence data may also be used during the enzyme selection step to both train models and provide a source of possible predicted sequences.

In embodiments, the server(s) 108 includes a reaction annotation engine 107 and a bioreachable prediction engine 109, which engines together or separately form the bioreachable prediction tool of embodiments of the disclosure. Alternatively, the software and associated hardware for the annotation engine 107, the prediction engine 109, or both may reside locally at the client 103 instead of at the server(s) 108, or be distributed between both client 103 and server(s) 108. The database(s) 110 may include public databases such as UniProt, PDB, Brenda, BKMR, and MNXref, as well as custom databases generated by the user or others, e.g., databases including molecules and reactions generated via synthetic biology experiments performed by the user or third-party contributors. The database(s) 110 may be local or remote with respect to the client 103 or distributed both locally and remotely. In some embodiments, the annotation engine 107 may run as a cloud-based service, and the prediction engine 109 may run locally on the client device 103. In embodiments, data for use by any locally resident engines may be stored in memory on the client device 103.

System Operation

Obtaining the Starting Metabolite Lists and Starting Reaction Data Sets

Inputs to the bioreachable prediction process include information such as starting metabolite list, starting reaction list, host cell, and baseline conditions, such as fuel level for the host (e.g., minimal or rich growth medium) and environmental conditions such as temperature. The annotation engine 107 may assemble metabolite and reaction data along with associated annotations from the database(s) 110.

Through the user interface 102, a user may specify the database(s) 110 from which to obtain information for the starting metabolite and reaction lists. For example, reactions and host-specific metabolites may be obtained from public databases such as KEGG, Uniprot, BKMR, and MNXref. (Those skilled in the art will recognize from the context of the discussion that references in this specification and the claims to “metabolites,” “reactions,” and the like may in many instances in fact refer to data representing those physical objects or processes and not the physical objects or processes themselves.)

Starting Metabolite List

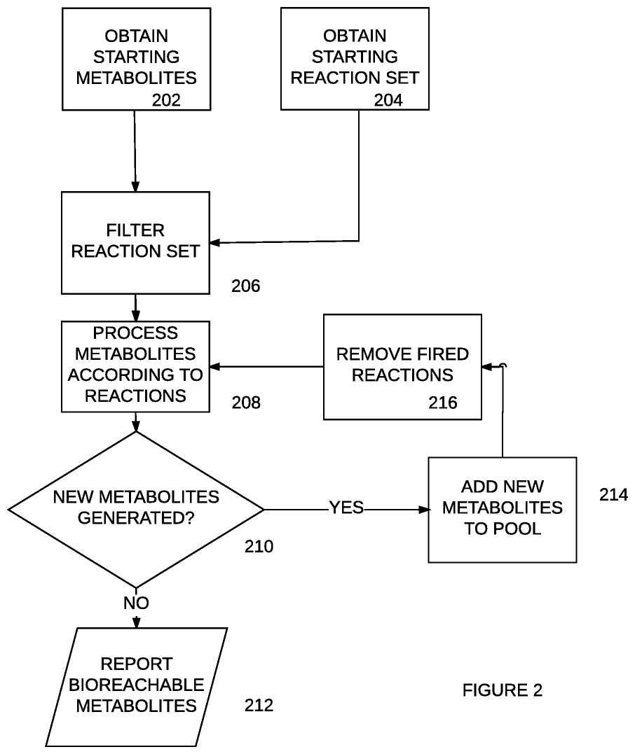

Referring to FIG. 2, in embodiments the reaction annotation engine 107 obtains or itself aggregates from the database(s) 110 a host-specific starting metabolite file comprising a list of chemical compounds (starting, intermediate, and final products) that are expected to be present during the growth of the host cell at a particular time or during a particular time interval under given growth conditions (202). The default growth condition may be a minimal growth medium, because this is the most conservative approach for selecting the starting metabolites. In embodiments, the reaction annotation engine 107 may provide the metabolite file as a starting metabolite list to the prediction engine 109.

In embodiments, the reaction annotation engine 107 may determine or template (off of similar microbes) the starting metabolites based on growth data for the host cell or for a similar cell. This approach is similar to approaches used to annotate the genomes of microbes in systems such as the RAST system, or to predict metabolic pathways in the BioCyc database collection. This approach uses the genome annotation for a given host cell to make a best guess at which metabolic pathways are present, and then assumes the presence of all the constituent reactions, and their metabolites, in those pathways. In the case of BioCyc databases, the existing genome annotation is used to identify the putative presence of individual enzymes (and thus their reactions). A rule-based system is then used to infer the presence of entire metabolic pathways based on the presence of (some of) their substituent reactions.

Having a starting metabolite list that is specific to the host cell is a distinguishing starting point for embodiments of the disclosure. Whereas other conventional approaches make generic predictions about targets that can be made, this customizable step of embodiments of the disclosure avoids the problem of making incorrect predictions about which target molecules can be made (or how they can be made) due to differences in the biology of host cells.

In embodiments, the user may instruct the reaction annotation engine 107 to retrieve the starting metabolites from existing databases or datasets, such as MNXref, KEGG or BKMR, based upon querying the databases or datasets with parameters such as host cell and growth medium, and, in some embodiments, via cross-indexing those databases with relevant model cell databases or other indications of the presence of specific metabolites. So far, for particular industrial hosts the assignees have created typical starting metabolite files on the order of 200-300 metabolites. As noted above, data objects representing metabolites in the public databases and the lists formed by the annotation engine 107 may include annotations including metadata such as host cell, growth medium type, and whether the metabolite is a core metabolite, a precursor, inorganic, or ubiquitous.

Core metabolites are the starting (e.g., substrate), intermediate and final metabolites natively found in a genetically-unmodified cells for given baseline conditions, such as the richness of the growth medium. Each core metabolite (e.g., amino acid) in the biomass of a microorganism like E. coli may be generated in the cell's core metabolism from one of eleven precursor metabolites, and may be fundamentally generated from whatever carbon input is provided to the genetically-unmodified cell. In embodiments, the user may select a starting metabolite set of select core compounds tagged with their precursor dependencies from databases such as MNXref, KEGG, ChEBI, Reactome, or others.

As their name suggests, inorganic metabolites, such as ammonium, do not include carbon, and thus cannot contribute a carbon atom to new products of the metabolism. Accordingly, the reaction annotation engine 107 may exclude inorganic metabolites from the starting metabolite set.

Some metabolites are ubiquitous, i.e., they are found in many reactions. They include molecules like ATP and NADP. Typically, ubiquitous molecules do not contribute carbon to the target product, and thus would not be part of any metabolic pathway to the target. Accordingly, the reaction annotation engine 107 may exclude ubiquitous metabolites from the starting metabolite set. Ubiquitous molecules can be manually designated in annotations based on expert evaluation or identified by determining what molecules participate in reactions beyond a particular threshold number. One heuristic flags all molecules that appear in the reaction set at numbers greater than the size of a typical core metabolite input (e.g., 300). For example, in one data set ATP appears in 2,415 of approximately 31,000 reactions, NADH appears in 2,000 reactions, and NADPH appears in 3,107 reactions, which places them above the core metabolite count and earns them all the “ubiquitous” tag.

Starting Reaction Data Set

The reaction annotation engine 107 obtains a starting reaction data set as the basis for prediction of viable target molecules (204). The user may specify how to build the starting reaction data set, or the user may instruct the annotation engine 107 to obtain the data directly from a public database 110 or a proprietary database 110, such as a custom database previously created by the user or others. In one embodiment, the annotation engine 107 may import the full reaction set (approximately 30,000 reactions) from the MetaNetx reaction namespace (MNX) of MNXref. In other embodiments, the annotation engine 107 may import and merge the reaction sets (approximately 22,000 total reactions) from MetaCyc and KEGG, or other public or private databases.

In embodiments, the reaction annotation engine 107 may build the starting reaction data set by selectively aggregating the information obtained from the database(s) 110. For example, BKMR provides information whether a reaction is spontaneous. The annotation engine 107 may use known mappings to map BKMR reaction IDs to IDs in MNXref for corresponding reactions. In other examples, KEGG or MetaCyc and their IDs may be employed instead of BKMR and its IDs. Using this association, the reaction annotation engine 107 may then create a custom reaction list in database(s) 110 using the existing annotations from MNXref (e.g., core, ubiquitous), along with a corresponding spontaneous reaction tag from BKMR. Similarly, through mapping corresponding IDs, the annotation engine 107 may associate reactions in MNXref with annotations in UniProt to obtain tags for whether a reaction is a transport reaction or whether a reaction substrate or product contains a halogen, and incorporate those tags into the annotations for the reaction in the custom reaction list in database(s) 110. (Identifying halogenated compounds is a heuristic for identifying reactions that run in the wrong direction, since most halogen-related reactions concern breaking down a chemical.)

Along these lines, the reaction annotation engine 107 may use associated IDs across databases to aggregate data from the databases to build a database 110 storing starting reaction sets with custom annotations, such as whether the reaction is spontaneous, runs in only one direction due to thermodynamics, contains a halogen (related to determining directionality), contains a ubiquitous metabolite, is a transport reaction, is unbalanced (that is, the two sides of the chemical reaction do not maintain elemental balance, suggesting the reaction is improperly written in the source database and should be ignored), is incompletely characterized in available databases, is associated with enzymes tagged with an indicator that the enzyme is associated with a known amino acid sequence or genetic sequence coding the enzyme, or is catalyzed by source enzymes likely to have transmembrane domains, among other tags. Through the annotation engine 107, the user may thus assign annotations to all of the approximately 30,000 reactions in the MNXref database, for example. As described below, the user may then configure criteria to filter this master file into individual lists for each annotation feature or any combination thereof.

Bioreachable Molecule Prediction

Referring to the flow diagram of FIG. 2, the following describes examples of the operation of the prediction engine 109 of embodiments of the disclosure. The prediction engine 109 predicts which chemicals can be created via, e.g., genetic engineering, in an arbitrarily selected host cell. The prediction engine 109 may take as inputs a starting metabolite file, a starting reaction data set, and a sequence database. The sequence database may store the amino acid sequences for catalytic compounds (such as enzymes), or the genetic sequences that encode catalytic compounds. Embodiments of the disclosure use the sequence database to determine the presence or absence of an amino acid sequence or genetic sequence for each reaction. In such embodiments, the sequence database need not include the sequences themselves, as long as the catalysts are tagged as having an enzyme or genetic part available or not. Along with a list of bioreachable candidate molecules, the prediction engine 109 produces for a specified host cell “pedigrees” (reaction pathways) of the reactions leading to production of each molecule from the starting metabolites, e.g., the host's core metabolites in some embodiments.

In particular, the predictions can be tuned based on a number of parameters, such as likely availability of catalysts to catalyze reactions, (e.g., likely availability of genetic parts to be engineered into the host cell or likely availability of catalysts to be introduced into the host cell via uptake from a growth medium in which the host cell is grown), maximum number of reaction steps allowed (starting from the starting metabolites), types of parts or chemical reactions to be allowed, and other selectable features. The prediction engine 109 also helps predict the approach to, and difficulty in designing target molecules by predicting the potential paths from core metabolites to each target molecule.

Filtered Reaction Data Set

In embodiments, the prediction engine 109 creates a filtered and validated reaction data set (RDS). Using the reactions characterized by the reaction annotation engine 107, the prediction engine 109 may filter the reactions to a desired level of validation, e.g., level of confidence that a coding sequence for the reaction enzyme exists (206). This is a step in fine tuning the accuracy of the predictions, and for controlling the primary source of false positive predictions. In an example mentioned above, the inventors generated the RDS for one bioreachable list by importing and annotating the full reaction set (approximately 30,000 reactions) from the MetaNetx reaction namespace (MNX) of MNXref. A similar approach could be applied to other publicly available reaction databases such as KEGG, Reactome, and MetaCyc.

Based upon the inventors' experience, 25-50% of the reactions in the most popular public databases may not have any known associated biological parts. For example, the amino acid sequences of enzymes for catalyzing the reactions, or their accompanying genetic sequences, may be unknown. Without the enzyme sequence information, a bioreactor would not be able to perform the reactions employing those enzymes, thus rendering the reaction information useless for engineering purposes. Even if only one enzyme within a pathway lacks a known gene sequence, then the entire pathway cannot be engineered into a host.

To deal with this deficiency, the prediction engine 109 may filter the reactions through a series of validation tests using publicly available or custom enzyme data. One public database is UniProt, which is large, open access, and reliably curated. Others include the RCSB Protein Data Bank (PDB) and GenBank. In some public databases such as MNXref, UniProt, Brenda, or PDB, reactions may be tagged with an Enzyme Commission (EC) number, which is a numerical classification for enzymes based on the reactions they catalyze. Some databases, such as UniProt or PDB, store EC number tags only for reactions for which the gene sequence coding the catalyzing enzymes are known. Other databases, such as KEGG and MetaCyc, include EC numbers for enzymes for which the gene sequence is not known.

Thus, depending upon the database, an EC number may or may not indicate the existence of a known enzyme gene sequence. Approximately, 20-25% of reactions with EC numbers have no associated enzyme coding sequence. In some cases, EC numbers are used to annotate multiple specific chemical transformations (there is a one-to-many relationship between EC numbers and chemical reactions), so that the presence of an enzyme sequence associated with an EC number does not mean that every reaction associated with that EC has a valid associated sequence. Thus, the presence of an EC tag on an enzyme activity is not a reliable general indicator of the presence of a gene sequence for that enzyme, but it can be applied to certain databases to determine if a sequence is reasonably likely to be present for that enzyme. Some databases also have separate fields (e.g. the “catalytic activity” field in UniProt) that explicitly describe particular chemical reactions as known to be definitively catalyzed by a given amino acid sequence (thus having a known genetic sequence for coding the enzyme catalyst). Such reactions are referred to herein as annotated as “definitively sequenced.”

The prediction engine 109 may determine a degree of confidence as to whether a catalyst is available to catalyze a reaction in the host cell (e.g., available to be engineered into the host cell to catalyze the reaction). For example, based on the differences in certainty that enzyme coding sequences are known, the prediction engine 109 may execute, in some embodiments, a “strict” search or a “relaxed” search for enzyme coding sequences against annotations in the reaction data set. For a strict search, the prediction engine 109 may select, for example, only reactions annotated as being definitively sequenced.

In embodiments, the prediction engine 109 may factor, into the degree of confidence as to whether a catalyst is available to catalyze a reaction, the degree of confidence (e.g., expect-value) that a sequence (e.g., enzyme amino acid sequence, nucleotide sequence) enables a desired function in a host cell, as described in embodiments below.

For a relaxed search, the prediction engine 109 may select, for example, reactions annotated as having an EC number that is associated with known enzyme coding sequences or (Boolean non-exclusive OR) reactions that are annotated as “definitively sequenced” in the sequence database, from annotations derived from databases such as MetaCyc. The prediction engine 109 records whether any gene or amino acid sequences are found for the reactions, for either level of confidence. For example, the prediction engine 109 may annotate the reaction with a tag indicating that it satisfies the relaxed search, but not the strict search.

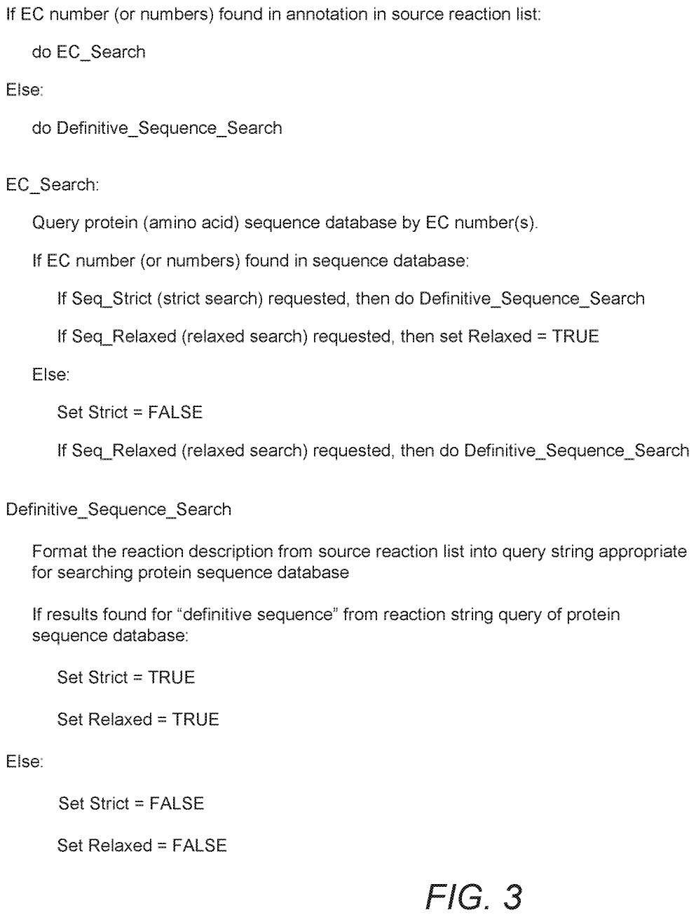

FIG. 3 illustrates exemplary pseudocode for implementing strict and relaxed enzyme sequence searches against databases, such as MNXref and UniProt, according to embodiments of the disclosure. The pseudocode describes the logic used by a heuristic for determining whether a sequence exists for an enzyme. This embodiment provides four levels of confidence. The code shows first determining whether the reaction data set annotations include at least one EC number. If so, then the code calls for searching the sequence database for EC numbers. If a strict search is being conducted, then the code calls for searching the sequence database for reactions that are definitively sequenced. If a relaxed search is being conducted, then the code sets the Relaxed annotation tag for the reactions having associated EC numbers to TRUE.

If the initial step determines that the reaction data set annotations (a) do not include an EC number or (b) (as mentioned above) the EC sequence search finds an EC number in the sequence database and a strict search is being conducted, then the code calls for searching the sequence database for reactions that are definitively sequenced. If that search finds a reaction as definitively sequenced, then the code sets both the Strict and Relaxed annotations for that reaction as TRUE. If not, then the code sets both those annotations for that reaction as FALSE.

In sum, the output of this heuristic are two annotation tags for each reaction: Strict and Relaxed. This heuristic provides four levels of confidence, as described below:

Strict=TRUE→very high confidence a sequence exists
Strict=FALSE→moderate confidence a sequence does not exist (expect some false negatives)
Relaxed=TRUE→moderate confidence a sequence exists (expect some false positives)
Relaxed=FALSE→very high confidence a sequence does not exist

The inventors have found that running a relaxed search results in less than a 20% false positive rate, whereas running a strict search against the catalytic activity field in UniProt results in a significant false negative rate. Thus, it may be better to err slightly on the side of a relaxed search. The “relaxed” and “strict” tags are just two potential methods of handling sequence-based filtering. The bioreachable prediction tool is amenable to any sequence-based tagging (and thus filtering) approach, including more permissive methods such as identifying the presence of sequences with appropriate motifs for the target activity or more stringent methods such as requiring the presence of a directly-literature-supported activity-sequence link in a heavily curated database such as MetaCyc.

As an alternative or in addition to the sequence-based filtering, the prediction engine 109 may filter (i.e., select or not select) reactions based upon any combination of the annotations discussed above with respect to the annotation engine 107, such as reaction directionality, or whether a reaction is a spontaneous reaction, a transport reaction, or contains a halogen. The prediction engine 109 may perform filtering based on user configuration through the user interface 102 or default settings. In embodiments, the prediction engine 109 may apply different filters in different reaction steps along the simulated metabolic pathways. As an example of default settings, they may be: reaction has a sequence based on relaxed criteria; exclude all transport reactions; only include reactions containing halogens if the reactions have a sequence; include all spontaneous reactions regardless of the above attributes.

If a reaction is spontaneous, the reaction will occur automatically without the need to engineer the host genome to produce an enzyme to catalyze the spontaneous reaction. Since the reaction is known to occur under given conditions for a given host, the prediction engine 109 can predict that the spontaneous reaction products will be produced.

As noted above, inorganic molecules do not contribute carbon and ubiquitous molecules are unlikely to contribute carbon to target metabolites. Thus, eliminating ubiquitous and inorganic molecules from those used as starting metabolites heuristically provides a high confidence level that the prediction engine 109 will follow valid metabolic pathways in predicting viable target molecules. Accordingly, the prediction engine 109 does not treat ubiquitous or inorganic molecules as limited in a reaction. That is, they are assumed to always be available to the reactions in which they participate.

Metabolite Prediction

Referring to FIG. 2, the prediction engine 109 may perform a stepwise simulation to predict which metabolites would be formed, given a substrate of input metabolites processed according to the reactions in the filtered RDS (208). (A chemical reaction operates on an input “substrate” (e.g., set of molecules) to produce chemical products.) The operation of the prediction engine 109 of embodiments of the disclosure may be described as follows:

Step 0: Initially, only core metabolites are present in the simulated host cell. They form the current substrate for the reactions in the next step.

Step 1: The prediction engine 109 determines whether the core metabolites from step 0 match one side of any of the chemical equations within the filtered reaction set (RDS), and whether a reaction can take place in a given direction (based on directional/thermodynamic annotation), to thereby determine which reactions would fire to produce chemicals on the other side of the reaction equation (208). The prediction engine 109 determines whether any new metabolites are produced by the fired reactions (210).

If the prediction engine 109 determines that no new metabolites have been predicted (210), then the prediction engine 109 ends the prediction process, and reports the results (212).

Conversely, if the prediction engine 109 determines that new metabolites would be formed (210), the prediction engine 109 adds the new metabolites to the substrate pool (214). The updated substrate pool now includes the core metabolites and the newly predicted metabolites from step 1.

The prediction engine 109 records the metabolites and fired reactions in each step, and also removes the fired reactions from the filtered RDS (step 216). This removal prevents the same reactions from being fired in subsequent steps, to thereby avoid a reaction and its resulting metabolite(s) from being identified as present in a subsequent step. Each reaction is simulated only once throughout all steps of the process. This comports with engineering best practices that generally focus on the shortest path (fewest number of steps) to reach a metabolite-longer pathways to the same metabolite are typically suboptimal. Along with the metabolites and reactions within each step, the prediction engine 109 records the step in which a metabolite is made (i.e., predicted to be made). That step represents the metabolic path length to generating the metabolite. Note that a metabolite may appear as a product in multiple steps if it is created via distinct reactions. This fact allows the prediction engine to identify usefully distinct pathways, where the same metabolite is reached by distinct reactions.

Step 2: The prediction engine 109 then returns to step 208 using the now updated substrate pool of metabolites as inputs to run against the filtered RDS (with fired reactions now removed) to predict whether any reactions would fire to produce new metabolites.

After multiple iterations, the pool of metabolites grows whereas the pool of available reactions shrinks. Eventually, the process may run to saturation because no more metabolites are left that can fire a reaction that remains in the filtered RDS. In experiments by the inventors, approximately 10,000 filtered reactions may result in several thousand metabolites after all iterations. Alternatively, the prediction engine 109 may be configured to specify the number of allowed reaction steps before halting the predictions and reporting the results (212). The limitation on number of reaction steps reflects real-world engineering, which would typically limit the number of cycles.

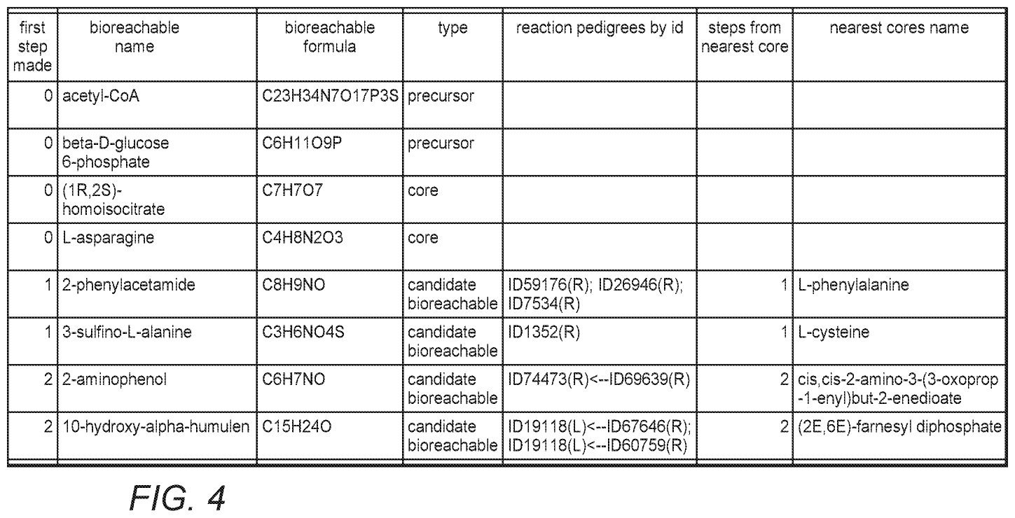

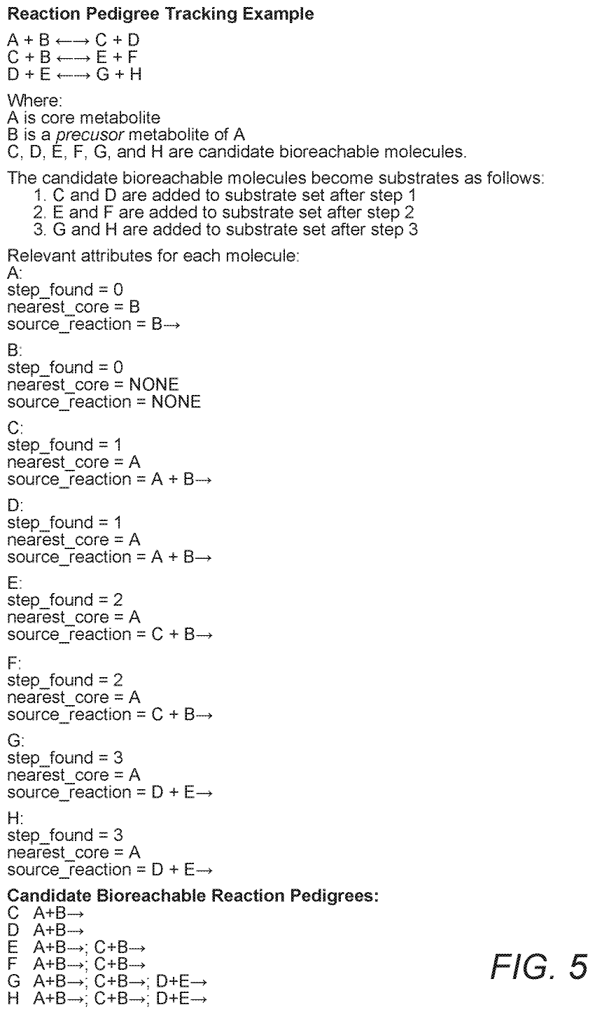

FIGS. 4 and 5 illustrate examples of reports that may be generated by embodiments of the disclosure. FIG. 4 shows, for each processing step, the metabolites generated (bioreachable name), their chemical formulas, the type of metabolite (e.g., core, precursor, candidate bioreachable produced by a reaction), the reaction pedigrees of the metabolites as denoted by a unique reaction ID such as an ID used in well-known databases (which also shows whether the left (“L”) or right (“R”) side of the reaction fired), the number of reaction steps needed from the nearest core metabolite to produce the candidate bioreachable molecule, and the name of the nearest core metabolite for each candidate bioreachable molecule. Note that the only molecules in step 0 are from the starting metabolite list (e.g., cores, precursors).

FIG. 5 illustrates a hypothetical example of reaction pedigree tracking. Stepwise the reactions are as follows:

Step 1: A+B← →C+D

Step 2: C+B← →E+F

Step 3: D+E← →G+H

The attributes in this example include: whether the metabolite generated in the step is a core; the step in which the metabolite is found; the nearest core metabolite to the generated metabolite, as measured by distance in number of steps; and the reaction pedigree denoting the chemical reaction fired to produce the metabolite. Metabolite A is a core metabolite and B is a precursor metabolite present in the biomass of the host at Step 0. Thus they have no reaction pedigree.

C and D are shown as produced in Step 1 by the reaction A+B in the reaction pedigree (source_reaction). The nearest core to both C and D is A. C and D are added to the substrate along with cores A and B.

E and F are shown as produced in Step 2 by the reaction C+B. The nearest core to both E and F is A. E and F are added to the substrate along with cores A and B and bioreachable products C and D.

G and H are shown as produced in Step 3 by the reaction D+E. The nearest core to both G and H is A.

Embodiments of the disclosure may also output the pathway (also known as the “pedigree” sequence of reactions) for each metabolite as follows:

C: A+B→

D: A+B→

E: A+B→; C+B→

F: A+B→; C+B→

G: A+B→; C+B→; D+E

H: A+B→; C+B→; D+E→

Pathway filtering. In embodiments, given a host cell, a target molecule, and the reaction pedigrees of the pathways leading to the given target molecule, the prediction engine 109 may selectively filter the pathways to identify pathways based on given parameters, such as path length (e.g., number of reaction processing steps from starting metabolite to target molecule). The prediction engine 109 may provide, as output, data representing the identified reaction pathways.

Host cell selection. Instead of determining viable target molecules given a single host cell, it may be desired to identify one or more host cells in which to produce a given viable target molecule. In embodiments, the prediction engine 109 generates data representing viable target molecules, according to methods described above, for not just one host cell, but for a plurality of host cells. In such embodiments, for a given viable target molecule, the prediction engine 109 determines at least one of the plurality of host cells that satisfies at least one criterion. For example, using the reaction pedigree data, the prediction engine 109 may select a host cell based upon the number of processing steps predicted as necessary to produce the given viable target molecule in that host cell. As another example, the prediction engine 109 may select a host cell based upon the predicted yield of the viable target molecule produced by that host cell. Predicted yield may be derived in a number of ways, including Flux-Balance Analysis (FBA) based on a separate model for each potential host, simple elemental yield modeling, and precursor-based percent yield estimates. The prediction engine 109 provides, as output, data representing the host cells determined to satisfy the at least one criterion.

As described for embodiments above, the prediction engine 109 may generate a record of one or more reaction pathways (i.e., pedigrees) leading to each target molecule produced by each host cell. Based on the above embodiments that run the tool for the plurality of host cells, the reaction annotation engine 107 may store associations between host cells, target molecules, and pedigrees in a database as a library, which may include annotations specifying parameters such as yield, number of processing steps, availability of catalysts to catalyze reactions in the reaction pathways, etc. Alternatively, the library may be obtained from a third party.

In embodiments, if the prediction engine 109 has access to such a library, the tool need not be run to identify multiple host cells in which to produce a given viable target molecule. Instead, in such embodiments, the prediction engine 109 may use the pedigrees from the library, which may include annotation data concerning associations among the hosts, target molecules, and reactions. The prediction engine 109 may identify at least one target host cell from among the one or more host cells based at least in part upon evidence, from, e.g., the library or public or proprietary databases, that all the catalysts predicted to catalyze reactions in at least one reaction pathway leading to production of the target molecule in the at least one target host cell are likely available to catalyze all such reactions in the at least one reaction pathway. In embodiments, the prediction engine 109 may determine target hosts based upon the target hosts requiring less than a threshold number of reaction steps within the reaction pathways that are predicted as necessary to produce the target molecule.

Bioprospecting. Some reaction enzymes may have an EC number and be well-characterized (their reactants and products are known), but not have a known associated amino acid sequence or genetic sequence (“orphan enzymes”). In such cases, the prediction engine 109 may bioprospect the orphan enzymes to predict their amino acid sequences, and, ultimately, their genetic sequences, so that the newly-sequenced enzymes may be engineered into the host cell to catalyze one or more reactions. The prediction engine 109 may then designate the reactions corresponding to the newly-sequenced enzymes as members of the filtered reaction data. In embodiments, the prediction engine 109 bioprospects the orphan enzymes using techniques known in the art. For example, one team determined the amino acid sequences for a small number of orphan enzymes by applying mass-spectrometry based analysis and computational methods (including sequence similarity networks and operon context analysis) to identify sequences. The team then used the newly determined sequences to more accurately predict the catalytic function of many more previously uncharacterized or misannotated proteins. Ramkissoon K R, et al. (2013) Rapid Identification of Sequences for Orphan Enzymes to Power Accurate Protein Annotation, PLoS ONE 8(12): e84508. doi:10.1371/journal.pone.0084508; see also Shearer A G, et al. (2014) Finding Sequences for over 270 Orphan Enzymes. PLoS ONE 9(5): e97250. doi: 10.1371/journal.pone.0097250; Yamada T, et al., Prediction and identification of sequences coding for orphan enzymes using genomic and metagenomic neighbours genomic and metagenomic neighbours, Molecular Systems Biology 8:581, all three of which are incorporated by reference in their entirety herein.

Genome engineering. Embodiments of the disclosure may be used to discover viable target molecules that have desired molecular properties or that contribute to related material properties. The bioreachable prediction tool may provide the list of bioreachable candidate molecules (viable target molecules) to a chemist, materials scientist or the like, who may be a third party such as a customer. Based upon their choice of target molecules, the user may instruct the tool to provide, to a gene manufacturing system, indications of the genetic sequences for the enzymes or other catalysts used to catalyze the reactions in the reaction pathways leading to each selected target molecule. The gene manufacturing system may then embody (through, e.g., insertion, replacement, deletion) the indicated genetic sequences into the genome of the host, to thereby produce an engineered genome for manufacture of the viable target molecules. In embodiments, the gene manufacturing system may be implemented using by systems and techniques known in the art, or by the factory 210 described in pending U.S. patent application Ser. No. 15/140,296, filed Apr. 27, 2016, published Nov. 2, 2017, entitled “Microbial Strain Design System and Methods for Improved Large Scale Production of Engineered Nucleotide Sequences,” incorporated by reference in its entirety herein. As described in that application, the gene manufacturing system may employ known techniques such as the Gibson and Golden Gate assembly protocols to assemble DNA sequences based upon input designs. The DNA constructs are typically circularized to form plasmids for insertion into a base strain. In the gene manufacturing system, the base strain is prepared to receive the assembled plasmid, which is then inserted. Input information may include techniques to employ during beginning, intermediate and final stages of manufacture. For example, many laboratory protocols include a PCR amplification step that requires a template sequence and two primer sequences. As is known in the art, the gene manufacturing system may be implemented partially or wholly using robotic automation. In embodiments, in addition to or as a substitute for embodying genetic sequences into the host, the prediction engine 109 provides to the factory an indication of one or more catalysts for the factory to introduce the one or more catalysts into the growth medium of the host cell for production of the target molecule.

Production of product of interest. Embodiments of the disclosure use well-known techniques to produce a viable target molecule or other product of interest from a base strain having a native or engineered genome. According to embodiments of the disclosure, the organism is transferred to a bioreactor containing feedstock for fermentation. Under controlled conditions, the organism ferments to produce a desired product of interest (e.g., small molecule, peptide, synthetic compound, fuel, alcohol) based upon the assembled DNA.

Different types of microbes can function as platform organisms in industrial biotechnology, including bacteria and yeasts fermenting sugar compounds into end-products, as well as microalgae via photosynthesis (phototrophic algae) or fermentation (heterotrophic algae).

The bacteria or other cells can be cultured in conventional nutrient media modified as appropriate for desired biosynthetic reactions or selections. Culture conditions, such as temperature, pH and the like, are those suitable for use with the host cell selected for expression, and will be apparent to those skilled in the art. Many references are available for the culture and production of cells, including cells of bacterial, plant, animal (including mammalian) and archaebacterial origin. See e.g., Sambrook, Ausubel (all supra), as well as Berger, Guide to Molecular Cloning Techniques, Methods in Enzymology volume 152 Academic Press, Inc., San Diego, Calif.; and Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelle et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue from Sigma-Aldrich, Inc (St Louis, Mo.) (“Sigma-LSRCCC”) and, for example, The Plant Culture Catalogue and supplement also from Sigma-Aldrich, Inc (St Louis, Mo.) (“Sigma-PCCS”), all of which are incorporated herein by reference.

The culture medium to be used should in a suitable manner satisfy the demands of the respective strains. Descriptions of culture media for various microorganisms are present in the “Manual of Methods for General Bacteriology” of the American Society for Bacteriology (Washington D.C., USA, 1981), incorporated by reference herein.

The synthesized cells may be cultured continuously, or discontinuously in a batch process (batch cultivation) or in a fed-batch or repeated fed-batch process for the purpose of producing the desired organic compound. A summary of a general nature about known cultivation methods is available in the textbook by Chmiel (Bioprozeltechnik. 1: Einfihrung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)), all of which are incorporated by reference herein.

Classical batch fermentation is a closed system, wherein the composition of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. Continuous fermentation generally maintains the cultures at a constant high density where cells are primarily in log phase growth. Continuous fermentation generally maintains the cultures at a stationary or late log/stationary, phase growth. Continuous fermentation systems strive to maintain steady state growth conditions.

Methods for modulating nutrients and growth factors for continuous fermentation processes as well as techniques for maximizing the rate of product formation are well known in the art of industrial microbiology.

For example, a non-limiting list of carbon sources for cellular cultures include, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

A non-limiting list of the nitrogen sources include, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

A non-limiting list of the possible phosphorus sources include, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate.

Finally, essential growth factors such as amino acids, for example homoserine and vitamins, for example thiamine, biotin or pantothenic acid, may be employed in addition to the abovementioned substances.

In some embodiments, the pH of the culture can be controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

The cultures may include an anti-foaming agent such as, for example, fatty acid polyglycol esters. The cultures may be modified to stabilize the plasmids of the cultures by adding suitable selective substances such as, for example, antibiotics.

The cultures may be carried out under aerobic or anaerobic conditions. In order to maintain aerobic conditions, oxygen or oxygen-containing gas mixtures such as, for example, air, are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation may be continued until an amount of the desired product of interest (e.g. an organic-chemical compound) sufficient for recovery has formed. This aim can normally be achieved within 10 hours to 160 hours. In continuous processes, longer cultivation times are possible. The activity of the microorganisms results in a concentration (accumulation) of the product of interest in the fermentation medium and/or in the cells of said microorganisms.

Pathway Prediction Examples

The prediction engine 109 may predict every pathway of reactions employing catalysts likely available to catalyze the reactions in the pathways or be engineered into the host to reach a target molecule, according to embodiments of the disclosure. The prediction engine 109 may also be used to select from among the predicted pathways to attempt manufacturing of the molecule based on qualitative information or quantitative information such as a score that may be generated by the prediction engine 109.

Reaction Labels and Categories

Reaction sets can be filtered and labeled as described elsewhere in this patent. For example, reactions can be labeled as “sequence relaxed,” to indicate they are likely to have gene sequences available, or they could be labeled as “characterized orphan” to indicate that genes exist in nature, but need to be experimentally characterized. Reactions can similarly be labeled to reflect their mass and energy balance, or other traits.

In addition, the bioreachable prediction tool may calculate in which direction a reaction is likely to operate based on thermodynamic data.

During processing of reactions to generate target molecules, the reaction annotation engine 107 can flag whether the production of a target molecule by a reaction happens in the thermodynamically favorable direction or in the thermodynamically unfavorable direction.

These thermodynamic results and all of the other reaction labels can then be used by the reaction annotation engine 107 to tag the molecules and pedigrees produced by a given run of the bioreachable prediction tool. For example, a five-step pedigree that contains one thermodynamically unfavorable reaction and two reactions lacking known genes to produce enzymes to catalyze the reactions could be labeled as:

Path length: 5

Unfavorable reactions: 1

Reactions lacking genes: 2

These labels then may be used by the prediction engine 109 to score each reaction. They also can be used to sort and operate on subsections of output, and they provide a direct insight into the engineerability of a given molecule for a given host.

In the examples detailed below, the bioreachable prediction tool was used to identify target molecules and display predicted pathways that may be used to reach those target molecules.

Thermodynamic data that was incorporated into pathway production and evaluation was generated using the group contribution method, but could also have been derived from any number of metabolic databases.

The prediction engine 109 may assign to each potential pathway an associated score created using the scoring method described herein. These scores can be used to inform decisions about which pathway variation to attempt to engineer to make the target molecule.

In embodiments, the prediction engine 109 may start with an optimal score of 100 points and subtract points for pathway features that add difficulty or risk of design failure. For example, path length correlates with design risk, and the total score may be reduced as path length increases, e.g., the prediction engine 109 may subtract from the score one or more points for each additional step in path length.

Tyramine

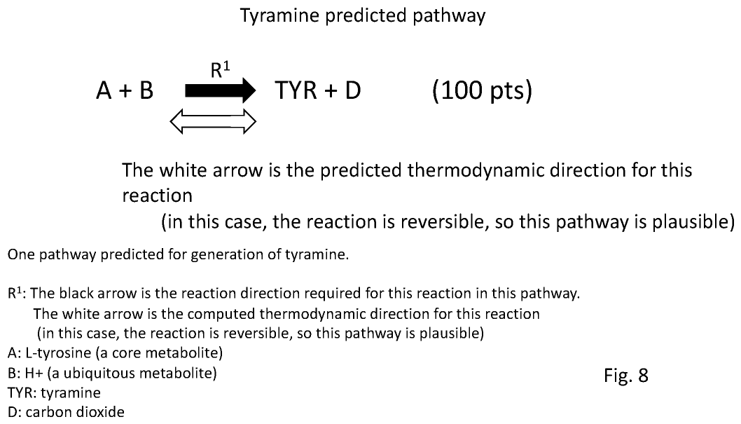

FIG. 8 illustrates a pathway identified by the prediction engine 109 to produce tyramine, according to embodiments of the disclosure. In the case of tyramine, a single pathway consisting of one reaction step (R¹) was predicted. The pathway shown depends on a reaction that is calculated based on thermodynamic data to be reversible, meaning it can operate in the direction required to generate tyramine.

In the pathway figures, a black arrow represents the reaction direction required for that reaction in the pathway to produce the desired molecule (here, tyramine). A white arrow represents the calculated thermodynamic direction for a reaction. When the required and calculated reaction directions match, the pathway is plausible.

This single pathway scores 100 points by the metric described elsewhere.

(S)-2,3,4,5-Tetrahydrodipicolinate (THDP)

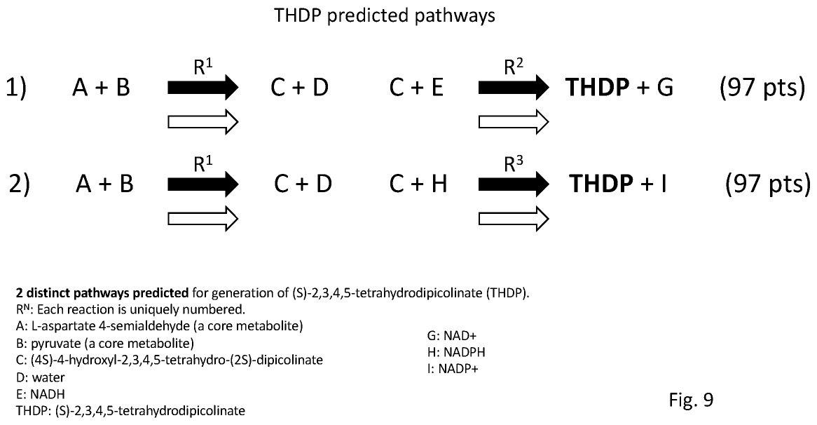

As shown in FIG. 9, the bioreachable prediction tool predicted two possible two-step pathways to generate THDP, according to embodiments of the disclosure. Both pathways achieve the same score of 97 points in these embodiments.

The pathways share the same first reaction (R¹) and differ at the second reaction (R² or R³). In this case, these reactions differ in which form of reducing cofactor they use, e.g., NADH versus NADPH. Although the pathways score the same, this cofactor difference is relevant for engineering purposes, and thus is displayed in this embodiment of the bioreachable prediction tool to help guide design decisions. Typically, one cofactor (either NADH or NADPH) is far more abundantly present in each given host cell. Thus, in embodiments, one skilled in the art may select the pathway employing the more abundant cofactor to produce THDP. In other embodiments, the prediction engine 109 may retrieve from a database and consider information concerning the influence of cofactors on engineerability to compute the target molecule score, thereby obviating the need for human review of the pathway cofactors.

Example Predicted Pathways for Hypothetical Molecule “F”

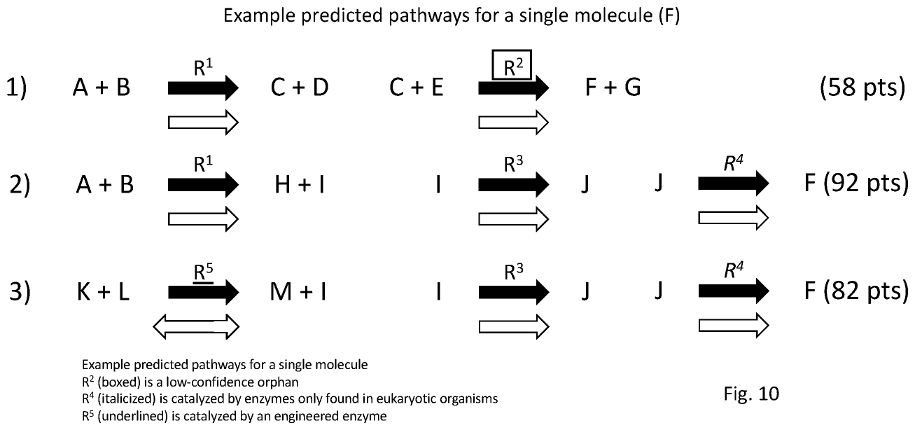

In another example, for a bioreachable molecule “F,” the bioreachable prediction tool has predicted three potential pathways, as illustrated in FIG. 10.

The first pathway is two steps long and includes a low-confidence orphan reaction (R²), leading to a score of 58 points. A low-confidence orphan reaction is a reaction catalyzed by an orphan enzyme for which it is unlikely that the corresponding DNA sequence is readily available without extensive, specific research work. Thus, many points are deducted for the orphan enzyme.

The second pathway is three steps long and includes one reaction with only eukaryotic genes available (R⁴), leading to a score of 92 points. Points are deducted because of overall pathway length and because of the limitation in sourcing genes for R⁴.

The third pathway is also three steps long and has two reactions (R³ and R⁴) in common with the other three-step reaction. It also has one reaction (R⁴) with only eukaryotic genes available and another reaction (R⁵) that requires an engineered enzyme, leading to a score of 82 points. In addition, this pathway has an alternate set of starting core metabolites (K+L instead of A+B) which has no impact on the pathway score, but is a consideration when deciding on which pathway is a best fit for the specific host and application.

In this example, the scoring output from the bioreachable prediction tool's prediction engine 109 provides critical engineering information beyond simple path length. Despite intuition that the shortest pathway (#1) might be best, information collected by the annotation engine 107 about each reaction and by the bioreachable prediction tool during filtering or processing show that the longer pathways (#2 and #3) might be more feasible to engineer. For example, the reaction annotation engine 107 may determine that catalysts for some reactions are only available in high-risk categories (e.g. low-confidence orphans, engineered enzymes), and the prediction engine 109 may determine that the short pathway depends on these high-risk categories whereas the long pathway does not, which may show that a longer pathway may be more feasible to engineer.

Tetrahydrodipicolinate Scoring Table

According to embodiments of the disclosure, the prediction engine 109 uses the information it generates to score the difficulty of producing target molecules. (Conversely, the score may be viewed as indicating the ease of producing molecules.) This score is interchangeably referred to herein as “molecule score,” “target molecule score,” or “overall pathway score.”

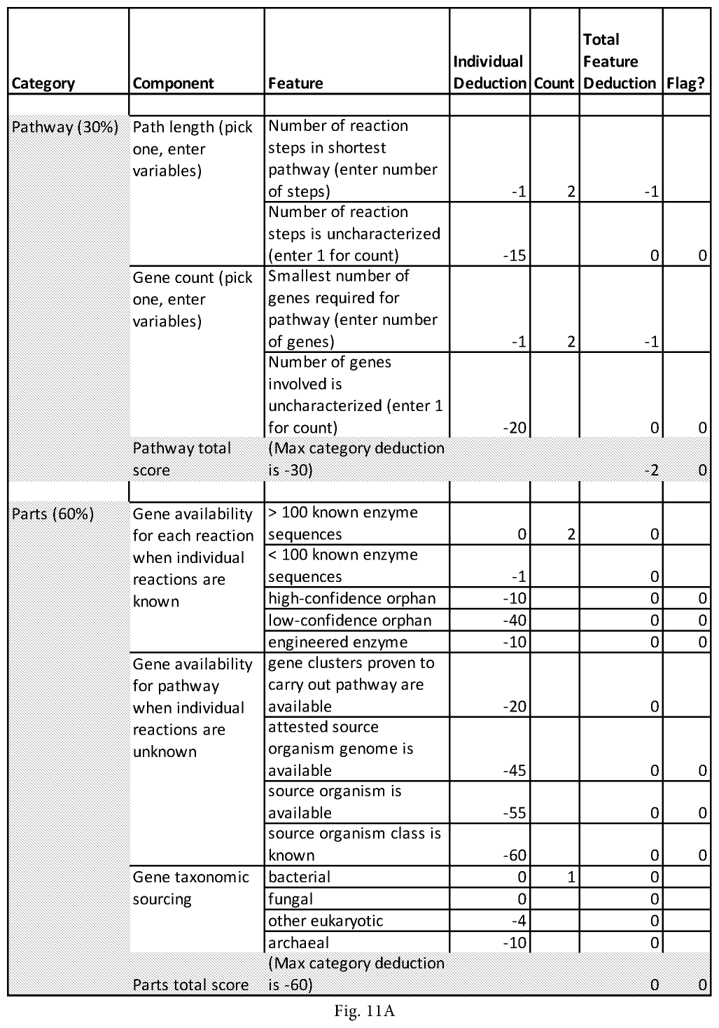

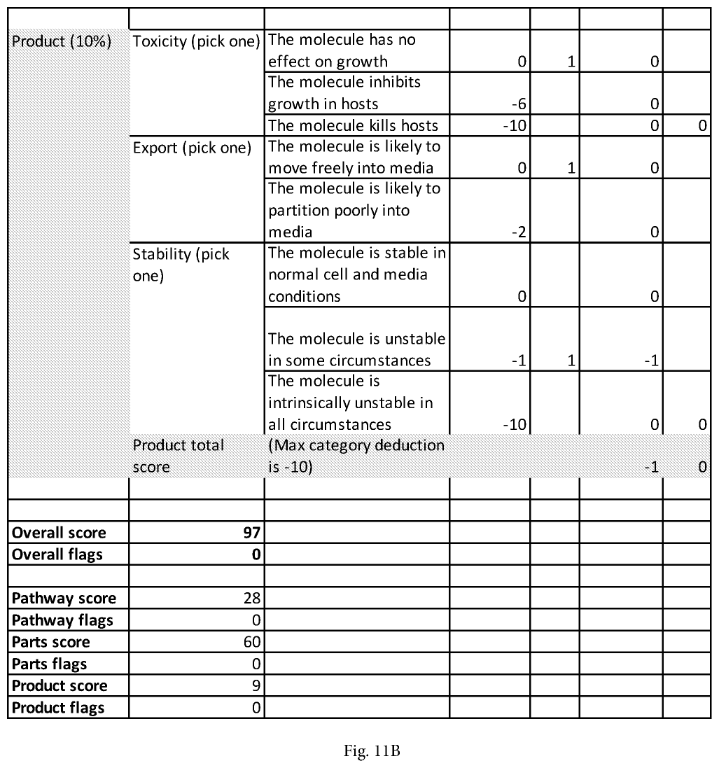

As an example, FIGS. 11A and 11B together provide a table illustrating how the prediction engine 109 may score the production of tetrahydrodipicolinate (THDP). In embodiments, the overall pathway scoring process may be broken down by components such as pathway score, parts score, and product score, weighted, e.g., as 30%, 60%, 10%, as shown in the table. The evaluation data shown was generated during the process of predicting pathways to the molecule (S)-2,3,4,5-tetrahydrodipicolinate (THDP).

Pathway component score represents the relative engineering feasibility of the pathway. In embodiments, it comprises two elements:

Path length—The number of reaction steps in the pathway. This is tallied as an intrinsic part of bioreachable prediction by the prediction engine 109, according to embodiments of the disclosure.

Gene count—The number of genes predicted to be required for the pathway. This is identified by querying databases as part of reaction filtering by the reaction annotation engine 107.

Since reactions and enzymes are not always in a 1:1 relationship (for example, a single reaction is sometimes catalyzed by a two-part enzyme, requiring two genes), the prediction engine 109 may factor both elements into the predicted difficulty of engineering the pathway.

In both pedigrees predicted by the bioreachable prediction tool, as shown in FIG. 9 THDP requires a two-step pathway in the desired host cell. This yields an appropriate score deduction based on the modest increase in difficulty of a 2- versus 1-step pathway.

In this case, the number of genes per pathway reaction step (identifiable via the same evaluation process that determines if a reaction is likely to have genes at all) also yields a modest penalty.

Parts Component Score

The Parts score represents the relative engineering feasibility of the individual pathway parts. In embodiments, it is based on the predicted difficulty in finding the parts (e.g., genes) required to engineer a catalyst into a host for the reactions in the pathway that is being evaluated.

In embodiments, the possible features that can impact the ability to find parts include:

>100 known enzyme sequences—100 or more sequences found for the reaction during the reaction filtering step (e.g., 100 or more amino acid sequences indicated in at least one database corresponding to enzymes for catalyzing the reaction)

<100 known enzyme sequences—enzyme sequences were found, but fewer than 100 were identified during the reaction filtering step

high-confidence orphan/low-confidence orphan—no enzyme sequences were found in public databases during the reaction filtering step, but associated evidence was found that suggested those sequences would be relatively easy (high-confidence) or hard (low-confidence) to identify

engineered enzyme—the only enzymes linked to this reaction during the reaction filtering step were engineered to carry out the reaction (this data can be found in database searches). This typically refers to natural enzymes that have been mutated to catalyze a reaction different from the reaction they naturally catalyze. These engineered enzymes can be difficult to use in novel pathways as they may be limited to one or a few sequences from a limited range of donor cells. Such engineered enzymes can be found in public databases such as BRENDA

gene taxonomic sourcing—also identified during the reaction filtering step (assuming enzyme sequences were found); this component classifies a candidate bioreachable molecule by the “worst case” (biggest penalty) among the reactions in the predicted pathway for that molecule; penalties are based on empirical data to date on the difficulty of expressing enzymes from the indicated sources in industrial platform cells

gene availability for pathway when individual reactions are unknown—in some cases pathways are defined using stand-in reactions in the dataset, and these reactions can be programmatically linked to individual gene clusters or cells; pathways in which individual reactions are unknown represent a significant increase in engineering risk and difficulty and thus a large penalty is assigned

These feature elements are all identified by the reaction annotation engine 107, as information is accumulated about the presence, absence, and abundance of sequence data for enzymes that catalyze each reaction.

In the case of THDP, genes are abundantly present for both pathway reactions, yielding no penalty. If instead, for example, one of the reactions were catalyzed by a low-confidence orphan, THDP would have accrued a significant penalty.

Product Component Score

The Product score is the smallest overall contributor to the target molecule score, in embodiments of the disclosure. The product score represents factors that influence the difficulty in sustaining the product in the cell, exporting it from the cell, and maintaining it in media. In embodiments, it represents an evaluation of the molecule's expected toxicity, exportability, and stability. The specific features described in this embodiment include:

Toxicity—The degree to which the molecule might be expected to be toxic to one or more host cells. This information can be derived from querying antimicrobial databases (or other databases that collect toxicity information on the general category of host cells).

Export—Predicted by querying chemical databases for partition coefficient data, or by querying internal experimental data.

Stability—Stability issues are identified by querying chemical databases.

Score Summary

The bottom of the table summarizes the overall score and the category scores. It also highlights any flags—areas that require particular derisking for pathway engineering. THDP happens to have no flags. An example flag would be if a pathway is missing one or more genes for its reaction steps (e.g., high- or low-confidence orphans).

Predicting Properties of Bioreachable Molecules and Related Molecules and Materials for Materials Innovation

Embodiments of the disclosure enable the determination of properties of bioreachable candidate molecules, non-bioreachable molecules derived from bioreachable candidate molecules, and materials that are related to bioreachable candidate molecules. The association of predicted material properties with related bioreachable candidate molecules allows for the efficient development of materials having desired properties, where a material can be synthesized starting with a “base” bioreachable candidate molecule.

### Glossary

To clarify, unless otherwise indicated herein, the term “molecule” refers to a type of molecule (e.g., a particular type of protein molecule), and not to an individual isolated molecule.

Similarly, to clarify, unless otherwise indicated herein, the term “cell” refers to a type of cell, and not to an individual isolated cell.

Unless otherwise indicated herein, the terms “actual bioreachable” molecule, “actually bioreachable” molecule, and “bioreachable” molecule are used interchangeably herein to refer to a molecule that can be produced in vivo, in vitro, or otherwise using one or more biological processes (e.g., bio-catalysis, transcription, translation).

Unless otherwise indicated herein, the term “candidate bioreachable molecule” or, interchangeably, “bioreachable candidate molecule,” refers to a molecule that is likely a bioreachable molecule. In embodiments, a candidate bioreachable molecule may be a molecule predicted to be a bioreachable molecule (e.g., in one or more given host cells) based on a set of starting metabolic reactions and metabolites. In embodiments, a candidate bioreachable molecule may likely be a bioreachable molecule that has not yet been confirmed to be bioreachable. In embodiments, a candidate bioreachable molecule may be a molecule stored in a database (e.g., database 110) for candidate or actual bioreachable molecules, but that has not yet been identified in the database as actually bioreachable. In embodiments, a candidate bioreachable molecule is a molecule with evidence (e.g., identified in a database) of being synthesized or isolated in a biological system (e.g., a single organism, or a consortium of multiple organisms or tissue types). A bioreachable candidate molecule may be a molecule suspected to be bioreachable because, for example, it has been predicted to be a viable target molecule using embodiments that are described in sections above. In embodiments, the term “candidate bioreachable molecules” includes the viable target molecules predicted by embodiments of the disclosure described above.

The term “putative bioreachable molecule” shall refer to an actual bioreachable molecule or a candidate bioreachable molecule.

Unless otherwise indicated herein, the term “material” shall include within its scope a single molecule or a combination of molecules for a given purpose (application), regardless of phase state.

Unless otherwise indicated herein, the term “physicochemical properties” shall include within its scope structural properties, including electronic structure.

“Chemical modeling” refers to modeling molecular properties based upon physicochemical properties, and includes electronic structure calculations (e.g., calculations based on first principles or related computational quantum chemistry methods such as DFT) or classical (i.e., non-quantum) simulations.

Operation

According to embodiments of the disclosure, the output of the Bioreachable Prediction Tool (BPT) described above and in the BPT PCT application is a list of possibly bioreachable molecules (bioreachable candidate molecules), and information about how to create them from biosynthetic reaction pathways.

Many of these molecules, however, have never been synthesized and there is limited or no information about their physicochemical properties. Embodiments of the disclosure extend the functional use and value of the BPT described above by creating a software-based system through which a user can rapidly identify specific bioreachable candidate molecules (e.g., from the BPT-generated list) that possess favorable physicochemical properties suitable for use as materials in a given application. The identification may be performed based on chemical properties predicted using electronic structure calculations (e.g., from first principles theories and related computational quantum chemistry methods such as density functional theory) or other chemical modeling methods (e.g., the simulation of molecular systems using molecular dynamics).

Embodiments of the disclosure rapidly and robustly identify bioreachable candidate molecules (or chemical derivatives thereof) for use in a material in a given application with minimal a priori information. Combined with embodiments/features of the bioreachable prediction tool described in the sections above and in the BPT PCT application, the prediction engine 109 may predict not only the biosynthesis of a given chemical, but also identify promising materials applications as well. Embodiments of the prediction engine 109 predict in silico bioreachable molecules that may not otherwise be identifiable via traditional synthetic routes or readily available commercially.

An example application is the prediction of bioreachable candidate molecules with the ideal chemical functionality and structure to form polymer films, which are prized for their chemical and mechanical strength, but face limited use due to less desirable optical features. The prediction engine 109 may calculate fundamental properties of precursor molecules (HOMO-LUMO gap, pKa, electronegativity, etc.) used to form useful film materials. Any relationships between calculated chemical properties and experimentally measured properties of merit (ease of formation, color, etc.) can be used to search actual or candidate bioreachable molecules stored in the chemicals database 110 to identify molecules with chemical properties that are highly correlated with promising film properties.

According to embodiments of the disclosure, the database 110 includes a chemicals database that stores information (both calculated and experimentally measured) about a set of bioreachable molecules (both actual and candidate bioreachable molecules). Examples of molecular properties include: molecular weight, density, electronic structure information (such as electron orbital energies), associated optical properties, solubility parameters, dipole moment, and number of accessible conformational states at room temperature. Second, the chemicals database 110 and associated servers 108 form a platform from which users may launch calculations (or simulations) to predict molecular properties in silico. Third, the chemicals database 110 enables physicochemical information stored in the database to be readily harvested for the construction of models (statistical, machine learning, et al.) used to predict materials properties.

Predicting Properties of Molecules and Materials

The bioreachable prediction tool (BPT) that is described in embodiments herein and in the BPT PCT application provides bioreachable candidate molecules and their reaction pathways, and stores them in the chemicals database 110, according to embodiments of the disclosure. In embodiments, the prediction engine 109 may use the bioreachable candidate molecules and their reaction pathways to predict their properties and properties of molecules and materials derived therefrom.

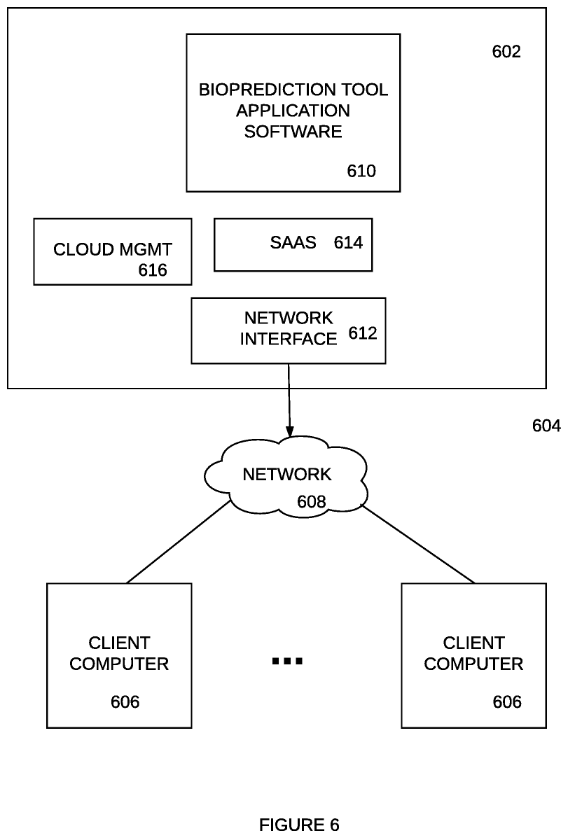

According to embodiments of the disclosure, the prediction engine 109 performs computational chemical calculations based on user instructions. The prediction engine 109 may receive the instructions from user interface 102 and employ cloud-based computational resources, or the SaaS system shown in FIG. 6. The prediction engine 109 may employ computer code that constructs input files necessary for the requested calculations based on existing data in the chemicals database 110 (e.g., chemical composition or ID), as well as stand-alone, executable packages (or containers) that can be easily deployed in different computer environments (mostly cloud-based systems) to perform computational chemical calculations. This arrangement enables the chemical property calculations to be performed at a scale commensurate with the size of the bioreachables list and on-demand. One potentially useful and less obvious consequence of this software engine is that calculations can be re-run when new computational methods are available in the future, such that calculated data stored in the chemicals database 110 can be periodically refreshed and updated on-demand.

Bioreachable Molecules

The prediction engine 109 may employ chemical modeling to predict properties of putative bioreachable molecules and properties of molecules derived from putative bioreachable molecules, according to embodiments of the disclosure. Computed molecular properties include: optimization of the geometry of a chemical molecule in the gas phase or implicit solvent, dipole moment, ionization potential, reduction-oxidation potential, acid dissociation constant (pKa), dielectric constant, chirality, electron bandgap, energy between the highest occupied molecular orbital and the lowest unoccupied molecular orbital (HOMO-LUMO gap), and spectroscopic properties (electronic or vibrational). The prediction engine 109 may write the predicted materials properties to the chemicals database 110.

Chemical modeling may, for example, be based on first principles or multiscale methods. Chemical modeling may focus on the foundational aspects of molecules derived from their electron structure, which can be calculated using techniques such as density functional theory (DFT). DFT is a quantum mechanical calculation that self-consistently and iteratively solves for the total energy and orbital energies of valence electrons in a given chemical system. Examples of software that has been developed to perform DFT and ab initio electronic structure calculations include NWChem and Gaussian 09, among others.

In addition to DFT, other chemical modeling methods that the prediction engine 109 may employ for predicting properties include computational calculations and simulations, such as: First Principles Calculations (FPC), Molecular Dynamics Calculations (MD), Monte Carlo Calculations (MC), Quantum Mechanics Modeling (QMM), or any combination thereof. Examples of chemical modeling software packages that employ these computational methods include LAMMPS (lammps.sandia.gov) and Biovia's Materials Studio.

In embodiments, the output of the chemical modeling may serve as input to a correlative model by, e.g., providing features on which machine learning can train to predict molecular properties.

Alternatively, the prediction engine 109 may employ correlative modeling (e.g., statistical modeling or machine learning) alone, without chemical modeling, to predict molecular properties, according to embodiments of the disclosure. An example statistical model is quantitative structure-activity relationship (QSAR) modeling, which need not rely on the output of the chemical modeling. QSAR produces predictive models derived from the application of statistical tools that correlate biological activity (or physicochemical properties in the case of QSPR chemical models) with descriptors representative of molecular properties (including structure).

Semi-Synthetic Molecules

Forward synthesis (described elsewhere herein) may be used in silico to predict molecules that may be derived from putative bioreachable molecules via chemical transformation. Because, in embodiments, the BPT without forward synthesis already may predict all bioreachable molecules of interest (subject to given constraints), the derivative molecules determined by forward synthesis will be semi-synthetic molecules. Such molecules are “semi-synthetic” in that their derivation starts with a putative bioreachable molecule followed by non-biological chemical transformation reaction steps.

For semi-synthetic molecules, the prediction engine may employ chemical modeling alone to predict semi-synthetic molecule properties, depending upon the nature of the derivative molecule (e.g., simple vs. complex). As an example, for a uni-molecular chemical system (e.g., neat solvents, small molecule drugs, bioactive molecules for cosmetics), chemical modeling alone may be sufficient, according to embodiments of the disclosure.

As with bioreachable molecules, the predictions may be enhanced with correlative modeling (e.g., machine learning) using the output of the chemical modeling of semi-synthetic molecules. Alternatively, the prediction engine 109 may employ correlative modeling alone to predict properties of semi-synthetic molecules, or correlative modeling followed by chemical modeling to (a) validate or refine one or more properties predicted by the correlative modeling, or (b) determine other properties, in manners similar to those described elsewhere herein concerning combinations of correlative modeling and chemical modeling.

Materials

Similar to the approach taken for certain semi-synthetic molecules, chemical modeling alone may be used to predict properties of a relatively small number of simple (e.g., uni-molecular) materials, such as neat solvents, small molecule drugs, bioactive molecules for cosmetics), according to embodiments of the disclosure.

Chemical modeling alone may also be used to predict the properties of materials that comprise in their chemical structure at least one bioreachable molecule or at least one semi-synthetic molecule derived from a bioreachable molecule, or a combination thereof, according to embodiments of the disclosure.

For these materials and many others, the prediction engine 109 may enhance the material properties predicted by chemical modeling with correlative prediction, according to embodiments of the disclosure. According to embodiments, this hybrid approach is particularly applicable to materials that comprise one or more precursor materials that undergo one or more chemical or physical processing steps (e.g., annealing, curing) to produce the final material (e.g., polymer films, chemically-derivatized interfaces). In embodiments, these materials are derived from putative bioreachable molecules, or from semi-synthetic molecules, which are themselves derived from putative bioreachable molecules.

In embodiments of material properties prediction, the output of the chemical modeling of the “base” putative bioreachable molecules, from which the materials are directly or indirectly derived, serves as input to the correlative modeling by, e.g., providing features on which the machine learning or the statistical model can train. In such embodiments, the prediction engine 109 may predict material properties such as density, solubility, glass transition temperature, mechanical properties, and viscosity.

In other embodiments, the prediction engine 109 may employ correlative modeling alone to predict properties of materials. In general, to predict properties of a material (or a semi-synthetic molecule), chemical modeling or correlative modeling, whether each alone or in combination, may be applied to the material (or semi-synthetic molecule) itself or to the base putative bioreachable molecule(s) from which the material (or semi-synthetic molecule) is derived. According to embodiments of the disclosure, for correlative modeling, such as machine learning, applied to predict material (or semi-synthetic molecule) properties, the model may be based upon training data of the materials (or semi-synthetic molecules).

Results from chemical modeling provide fundamental chemical properties that are known to influence chemical structure and reactivity of a bioreachable molecule and, in some instances, of materials derived from such a molecule. The use of computational chemical calculations to guide efficient materials innovation is known in the electronics industry, where electronic structure calculations have been used to rapidly and cheaply screen candidate materials for applications in batteries and optoelectronics in silico (see The Materials Project for an example).

Forward Synthesis

Forward synthesis may be used to predict molecules derivable from putative bioreachable molecules (e.g., predict semi-synthetic molecules). Embodiments of the disclosure provide a computer-aided synthesis design tool to enable forward synthesis starting from a putative bioreachable molecule. This tool may reside in the prediction engine 109. The design tool enables the expansion of the database to a much larger chemical space that can be quickly reached by starting with the putative bioreachable molecule and performing a finite number of chemical synthesis steps in silico. This tool supports material innovation because the ideal molecule for an application may not be one of the putative bioreachable molecules, but may be a highly related “neighboring” molecule derived from a putative bioreachable molecule. The prediction engine 109 may store the results of forward synthesis in database 110. The prediction engine 109 may use these results to determine whether a given molecule is derivable from a “base” putative bioreachable molecule. As described elsewhere herein, the prediction engine 109 may predict the properties of the molecules derived via forward synthesis (e.g., semi-synthetic molecules).

These neighboring molecules may still be too expensive to produce through normal synthesis methods, but may be commercially viable if the putative bioreachable molecule can be used as a starting point for a limited number of synthesis steps, e.g., two or three steps (e.g., hydrogenation). Note that within just two steps, useful molecules may be produced that may be used by themselves or in combination with other molecules to create interesting materials. For example, a putative bioreachable molecule to which two synthesis steps are applied may be combined with other molecules to form a film.

These neighboring molecules from the computer-aided synthesis design tool may also have never been built and tested, and thus can be added to the chemicals database 110, annotated by the annotation engine 107 with calculated property information, and searched, like the putative bioreachable molecules.

Stand-alone implementations of the computer-aided synthesis design tool are sometimes referred to as “computer-assisted organic synthesis” tools. According to embodiments of the disclosure, the prediction engine 109 may combine these tools with the other modules described herein to enable materials innovation with putative bioreachable candidate molecules or molecules that are chemically derived from putatative bioreachable molecules. Examples of computer-aided synthesis design tools include the following well-known software packages: Chematica, ChemPlanner, ICSynth, and WODCA, among others.

Due to the large number of potential synthesis transformations which can be applied to a molecule, forward synthesis can dramatically increase the size of the chemicals database. Such transformations include chemical transformations (e.g., hydrogenation, condensation, composition, polymerization). Any one molecule can be potentially transformed into hundreds or thousands of neighboring molecules, and repeating this process just a few times can turn a short list of bioreachable molecules into database of hundreds of millions of molecules. Note that even two steps performed on 2000 bioreachable molecules results in an enormous output-approximately 2×10\hat{ }7 molecules. This potential explosion of database size motivates the need for the automated and highly scalable systems for chemical property calculations described in this disclosure.

Combinations of Correlative Modeling and Chemical Modeling

Because chemical modeling can consume relatively large amounts of computational time compared to correlative modeling, chemical modeling may be reserved as an optional step for later validation of the predicted properties. According to embodiments of the disclosure, the prediction engine 109 identifies a first set of putative bioreachable molecules (or semi-synthetic molecules or materials) that possess a desired property based on associations between (a) putative bioreachable molecules (or semi-synthetic molecules or materials) and (b) experimentally-determined properties or predicted properties (or both). According to embodiments of the disclosure, those predicted properties are determined via correlative modeling (e.g., machine learning or statistical modeling).

According to embodiments of the disclosure, the prediction engine 109 may rank or score this identified first set of putative bioreachable molecules (or semi-synthetic molecules or materials) based on factors such as ease of engineering, number of reaction steps, availability of parts, or other factors such as those described herein with respect to molecule/overall pathway scoring.

For those putative bioreachable molecules (or semi-synthetic molecules or materials) of the first set satisfying a quality threshold (e.g., a score or rank threshold such as the top three molecules with the shortest pathways), the prediction engine 109 employs chemical modeling to confirm the properties predicted by the correlative modeling, according to embodiments of the disclosure. For example, for putative bioreachable molecules, and for simple semi-synthetic molecules and simple materials of the first set that are amenable to chemical modeling (e.g., uni-molecular materials), the prediction engine 109 may employ chemical modeling to determine whether the properties predicted by the computationally intensive chemical modeling match the correlatively modeled properties of some, all or none of the bioreachable molecules (or semi-synthetic molecules or materials) predicted by correlative modeling to possess the desired properties. The user or the prediction engine 109 may select the matching set of the putative bioreachable molecules (or semi-synthetic molecules or materials) for display and for further exploration or production. The selection may, for example, be based upon satisfaction of a rank or threshold relating to the match or to one or more desired qualities. See Example 3 below.

According to embodiments of the disclosure, for the molecules or materials of the first set predicted by correlative modeling, the prediction engine 109 employs correlative modeling or chemical modeling to predict other properties of those molecules or materials.

According to embodiments of the disclosure, the prediction engine 109 may rank or score the molecules or materials of the first set based on factors such as ease of engineering, availability of parts, or other factors such as those described herein with respect to molecule/overall pathway scoring. The user or the prediction engine 109 may select, from those molecules or materials of the first set, a second set of molecules or materials that correspond to experimentally determined properties or properties predicted by the same or a different correlative model or by chemical modeling (or a combination thereof) for display and further exploration or production. The selection may, for example, be based upon satisfaction of a rank or threshold relating to one or more desired properties. See Examples 1 and 2 below.

Identifying Molecules or Materials Based Upon Desired Properties and Ease of Producing Molecules or Materials

As shown in FIG. 5, the recorded pedigree may include a record of the reaction step in which each viable target molecule is predicted to be generated, as well as the nearest core metabolite. FIGS. 11A-11B show an example of pathway scoring broken down into recorded components that contribute to pathway score, parts score, and product score.

Thus, in embodiments of the disclosure, the search for viable target molecules (or related semi-synthetic molecules or materials) based on desired properties (as described elsewhere herein) may be further constrained by parameters representing the ease (or difficulty) of producing target molecules, including, e.g., the distance between a predicted target molecule and a core metabolite (e.g., nearest core metabolite). The parameters representing the ease (or difficulty) of producing target molecules may represent one or more components contributing to the pathway score, or the overall pathway score itself or one or more of its constituent components.

For example, the prediction engine 109 may determine and record in a database the distance, measured in steps, between any two viable target molecules (e.g., ancestor and descendant) along the same reaction pathway as the difference between the step numbers in which the molecules are generated. For example, in FIG. 5 molecule C is generated in reaction step 1, and molecule H is generated in step 3. The distance between C and H is 2. According to embodiments of the disclosure, a user may query which viable target molecules satisfy desired molecular or material properties and are also within three reaction steps of a viable target molecule that the user knows may be easily and inexpensively produced.

According to embodiments of the disclosure, a user may want to know which viable target molecules satisfy desired molecular or material properties where each such molecule is produced by one or more reactions that are indicated (e.g., in a database) as catalyzed by one or more corresponding catalysts that are themselves indicated as available to catalyze the reactions. According to embodiments of the disclosure, a user may want to know which viable target molecules satisfy desired molecular or material properties where each such molecule is determined by whether it is produced by one or more reactions that are indicated (e.g., in a database) as catalyzed by one or more corresponding catalysts that are themselves indicated as corresponding to one or more amino acid sequences or one or more genetic sequences. According to embodiments of the disclosure, a user may want to know which viable target molecules satisfy desired molecular or material properties where each such molecule is produced by one or more reactions that are indicated (e.g., in a database) as catalyzed by one or more corresponding catalysts that are themselves indicated as able to be engineered into an organism or taken up from the growth medium in which an organism is grown.

Embodiments of the disclosure extend this searching capability to materials by enabling searching for semi-synthetic molecules or materials based upon desired properties (as described elsewhere herein) as well as the ease (or difficulty) of producing molecules that constitute simple (e.g., uni-molecular) materials such as those described elsewhere herein, or base molecules from which semi-synthetic molecules or materials are directly or indirectly derived.

### EXAMPLES

### Example 1: Identify Bioreachable Molecules (“BRMs”) Having Desired Properties

Prediction of Water-Soluble BRMs

Initial Query Criteria:
Desired property:
   Soluble in water: Yes
Contains the following chemical reactive groups:
   at least one primary amine group
   at least one primary alcohol group
   at least one phenyl group

Secondary Selection Criteria:
Desired Property:
   Dipole Moment: 2-3 Debye

Primary Selection Step Using Correlative Modeling:

An initial search of a database of bioreachable molecules (“BRMs”) identified all putative BRMs that satisfied the criteria for predicted solubility in water as well as that for containing unique chemical reactive groups (at least one primary amine group, at least one primary alcohol group, and at least one phenyl group). In this case, solubility was predicted using a correlative model, a machine learning based approach to predict Hansen solubility parameters using Gaussian processes, to predict solubility in the solvent water. All BRMs for which the mean square sum of the predicted solubility parameters (6) were found to be less than 8 MPa^{1/2} of that predicted for water were predicted to be chemically similar enough to water and, therefore, soluble. In this example, this search returned a set putative BRMs comprising 3 structures.

Secondary Selection Step Using Chemical Modeling:

A secondary selection step was performed to identify which of the putative BRMs in the returned set of BRMs have a predicted electric dipole moment within the range of 2-3 Debye. Dipole moment serves as a measure of molecular polarity and is a useful predictor of molecular response to external applied electric fields. In this step, each of the putative BRMs resulting from the initial step were submitted to a density functional theory calculation for the prediction of the dipole moment of the lowest energy molecular configuration for each of the putative BRMs from the initial step. In this example, the putative BRM tyramine, with a dipole moment of 2.45 Debye, was found to be the best candidate BRM that met the criteria specified in both the primary and secondary selection steps.

### Example 2: Identify BRMs that Produce Materials Having Desired Properties

Identification of BRMs that Produce a Non-Toxic Solvent (Unimolecular Material)

### Initial Query Criteria:

Desired property:
   Soluble in N-methylpyrollidone: Yes
Contains the following chemical reactive groups:
   at least one primary amine group
   at least one primary alcohols group
   at least one phenyl group

Secondary Selection Criteria:
Desired Property:
   Non-toxic

Primary Selection Step Using Correlative Modeling:

An initial search of the BRM database identified all putative BRMs that satisfy the criteria for predicted solubility as well as that for unique chemical reactive groups (at least one primary amine group, at least one primary alcohol group, and at least one phenyl group). In this case, solubility in the solvent N-methylpyrollidone of a unimolecular material composed of a BRM (or a molecule related to the BRM by chemical transformation) was predicted using a correlative model, a machine learning based approach to predict Hansen solubility parameters using Gaussian processes. All unimolecular materials composed of BRMs for which the mean square sum of the predicted solubility parameters (6) were found (for the unimolecular materials) to be less than 8 MPa^{1/2} of that predicted for N-methylpyrollidone were predicted to be soluble. For the purposes of rapid screening of candidate BRMs, unimolecular BRM materials with the highest solubility in N-methylpyrollidone are assumed to be good candidates to replace N-methylpyrollidone as a solvent in industrial applications. This search returned a set comprising putative BRMs for which the predicted solubility of the corresponding unimolecular material satisfied the primary selection criteria.

Secondary Selection Step Using Correlative Modeling:

A secondary selection step was performed to identify which putative BRMs in the returned set of BRMs are predicted to be non-toxic. In this step, a machine learning model approach based on a 2-layered deep neural network was used to predict the toxicity of each of the putative BRMs resulting from the initial step for 12 different toxic effects. In this example, the application of the second step, based on the assumption that the toxicity predicted for an isolated BRM will also be similar to that of a corresponding unimolecular material, further reduced the set of putative BRMs from 7 to 4.

### Example 3: Identify BRMs that can Produce a Material with Desired Properties

Identification of Polymer Composed of BRMs with High Glass Transition Temperature

Primary Selection Step:
Desired property #1:
   Glass transition temperature >200 degrees C.
Desired property #2:
   Max. number of biosynthetic reaction steps from core metabolite for a given host: 1
Contains the following chemical reactive groups:
   at least one primary amine group
   at least one primary alcohols group
   at least one phenyl group

Secondary Refinement Step:
Desired Property:
   Glass transition temperature >200 degrees C.

Primary Selection Step Using Correlative Modeling:

An initial search of the BRM database identified all putative BRMs that could serve as at least one constituent component of a polymer that satisfied the criteria for predicted glass transition temperature (Tg). The search criteria also included additional requirements for the putative BRM, from which the putative polymer material would be derived: presence of unique chemical reactive groups (at least one primary amine group, at least one primary alcohol group, and at least one phenyl group) and ease of bioreachability (e.g., number of biosynthetic steps from core metabolite). Tg values were predicted for a group of polymers containing BRMs using a QSPR based approach based on chemical connection indices and experimentally measured Tg. This search returned a set comprising 7 polymers based on putative BRMs with Tg values that met the Tg criteria. Note that the predicted Tg values from the correlative model have an associated average error of >15%.

Secondary Refinement Step Using Chemical Modeling:

In this secondary refinement step, atomistic molecular dynamics simulations were carried out for the set of BRM-based polymers returned from previous step. The purpose of this step was to predict Tg values with higher accuracy. In this case, one of the target BRM-based polymers was a polymer based on tyramine. The predicted Tg value from the machine learning model was 209+/−34 degrees C. In contrast, the predicted Tg value from molecular dynamics simulations was higher and had better accuracy: 301+/−10 degrees C. This refinement step allows scientists to further contract or expand the set of BRM-based polymers using more reliable predicted data.

User Interface

According to embodiments of the disclosure, software at server 108 enables the user at user interface 102 to interface with the chemicals database (included in database 110) to view identifiers of the bioreachable candidate molecules along with their predicted properties to determine whether a molecule may be suitable for a given application. The interface allows the user to visually inspect existing chemical information (chemical structure or properties), launch computational chemical calculations or simulations, as well as use existing predictive models for materials behavior to assess the potential value of a given target molecule.

Machine Learning

Embodiments of the disclosure may apply machine learning (“ML”) techniques to learn the relationship between the given parameters (features) and observed outcomes (e.g., experimental data concerning molecule or material properties). In this framework, embodiments may use standard ML models, e.g. Decision Trees, to determine feature importance. In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes exhibited by training data. The result of the learning is then used to predict whether new data will exhibit the same patterns, categories, statistical relationships or other attributes.

Embodiments of this disclosure may employ unsupervised machine learning. Alternatively, some embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) or Tensor processing units (TPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

Computer System Implementation

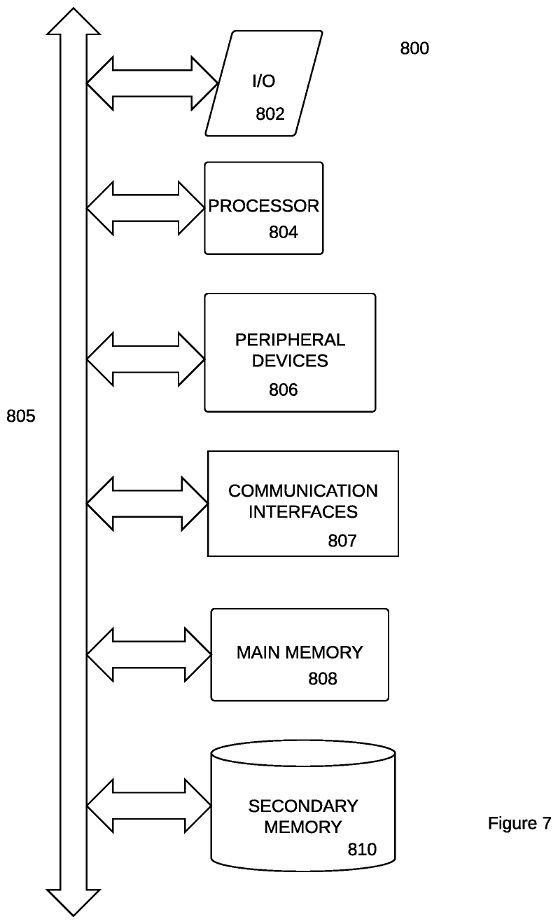

FIG. 6 illustrates a cloud computing environment 604 according to embodiments of the present disclosure. In embodiments of the disclosure, the software 610 for the reaction annotation engine 107 and the prediction engine 109 of FIG. 1 may be implemented in a cloud computing system 602, e.g., to enable multiple users to annotate reactions and predict bioreachable molecules according to embodiments of the present disclosure. Client computers 606, such as those illustrated in FIG. 7, access the system via a network 608, such as the Internet. The system may employ one or more computing systems using one or more processors, of the type illustrated in FIG. 7. The cloud computing system itself includes a network interface 612 to interface the bioreachable prediction tool software 610 to the client computers 606 via the network 608. The network interface 612 may include an application programming interface (API) to enable client applications at the client computers 606 to access the system software 610. In particular, through the API, client computers 606 may access the annotation engine 107 and the prediction engine 109.

A software as a service (SaaS) software module 614 offers the BPT system software 610 as a service to the client computers 606. A cloud management module 616 manages access to the system 610 by the client computers 606. The cloud management module 616 may enable a cloud architecture that employs multitenant applications, virtualization or other architectures known in the art to serve multiple users.

FIG. 7 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the annotation engine 107 and the prediction engine 109, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, the elements of bioreachable prediction tool and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 6.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they are disclose essential matter.

In the claims below, a claim n reciting “any one of the preceding claims starting with claim x,” shall refer to any one of the claims starting with claim x and ending with the immediately preceding claim (claim n−1). For example, claim 35 reciting “The system of any one of the preceding claims starting with claim 28” refers to the system of any one of claims 28-34.

### Embodiments

Each embodiment below corresponds to one or more embodiments of the disclosure. It is understood that the use of the term “claim” in this section refers to an “embodiment” and not to a claim of this application. Dependencies below are understood to refer back to embodiments within the same set.

### Methods

### Set M1A Predicting Properties of Bioreachable Molecules

1. A method for predicting at least one property of a first molecule of one or more putative bioreachable molecules, the method comprising:
   generating a chemical model of the first molecule based on physicochemical properties; and
   predicting the at least one property of the first molecule based at least in part upon the chemical model and empirical data concerning the first molecule, wherein the empirical data does not include data concerning the at least one property.
2. The method of claim 1, wherein predicting the at least one property of the first molecule employs statistical modeling or machine learning.
3. The method of claim 1, further comprising obtaining the first molecule.
4. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set M1B Query Database to Identify BRM with Desired BRM Properties
1. A computer-implemented method for identifying a putative bioreachable molecule having a desired property, the method comprising:
   a. receiving a query of a database, the query indicating a desired property, wherein:
      i. the database stores associations between one or putative bioreachable molecules and one or more predicted properties of the one or more putative bioreachable molecules, and
      ii. the one or more predicted properties include the desired property; and
   b. returning data representing a first putative bioreachable molecule of the one or more putative bioreachable molecules based at least in part upon association of the first putative bioreachable molecule with the desired property.
2. The method of claim 1, wherein the one or more predicted properties are based at least in part upon chemical modeling of the one or more putative bioreachable molecules.
3. The method of any one of claim 1 or 2, wherein the one or more predicted properties are based at least in part upon empirical data concerning the one or more putative bioreachable molecules.
4. The method of claim 1, wherein the one or more predicted properties are based at least in part upon statistical modeling or machine learning.
5. The method of claim 1, further comprising obtaining the first putative bioreachable molecule.
6. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set M2A Predict Material Properties Based Upon Related Bioreachable Molecule

1. A computer-implemented method for predicting at least one property of a material related to a first molecule of one or more putative bioreachable molecules, the method comprising:
   a. generating a chemical model of the material based on physicochemical properties; and
   b. predicting the at least one property of the material based at least in part upon the chemical model and correlative modeling.
2. The method of claim 1, wherein predicting the at least one property of the material is based at least in part upon predictive modeling of the first molecule.
3. The method of claim 1, wherein the material comprises at least the first molecule.
4. The method of claim 1, wherein the material comprises at least two instances of the first molecule in its structure.
5. The method of claim 1, wherein the material comprises in its chemical structure at least the first molecule or at least one semi-synthetic molecule derived from the first molecule, or a combination thereof.
6. The method of claim 1, further comprising obtaining the material.
7. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set M2B Query Database to Identify a Material with Desired Material Property
1. A computer-implemented method for identifying a material having a desired material property, wherein the material is related to one or more putative bioreachable molecules, the method comprising:
   a. receiving a query of a database, the query indicating a desired material property, wherein:
      the database stores associations between one or more materials and one or more predicted material properties of the one or more materials, and
      the one or more predicted material properties include the desired material property,
      the one or more predicted material properties are based at least in part upon chemical modeling of the one or more putative bioreachable molecules; and
   b. returning data representing a first material of the one or more materials based at least in part upon association of the first material with the desired material property.
2. The method of claim 1, wherein the associations are based at least in part upon statistical modeling or machine learning.
3. The method of claim 1, wherein the first material comprises in its chemical structure at least one bioreachable molecule of the one or more putative bioreachable molecules or at least one semi-synthetic molecule, or a combination thereof.
4. The method of claim 1, further comprising obtaining the first material.
5. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set M3A Query DB to Identify BRM that can be Used to Produce Material with Desired Material Property
1. A computer-implemented method for identifying one or more putative bioreachable molecules related to a desired material property, the method comprising:
   a. receiving a query of a database, the query indicating a desired material property, wherein the database stores associations between one or more putative bioreachable molecules and material properties of one or more materials; and
   b. returning data representing one or more putative base bioreachable molecules of the one or more putative bioreachable molecules,
      i. wherein the one or more putative base bioreachable molecules are related to at least one material, of the one or more materials, that has the desired material property.
2. The method of claim 1, wherein the at least one related material includes within its structure the one or more putative base bioreachable molecules.
3. The method of claim 1, wherein the at least one related material is a polymer and each putative base bioreachable molecule of the one or more putative base bioreachable molecules is a monomer.
4. The method of claim 1, wherein the at least one related material comprises in its chemical structure at least one base bioreachable molecule of the one or more putative base bioreachable molecules or at least one semi-synthetic molecule, or a combination thereof.
5. The method of claim 1, wherein the association between the one or more putative base bioreachable molecules and the desired material property is based at least in part upon predictive modeling.
6. The method of claim 6, wherein the predictive modeling employs statistical modeling or machine learning.
7. The method of claim 1, wherein the returned data includes data representing at least one reaction pathway between the one or more putative base bioreachable molecules and the at least one related material.
8. The method of claim 1, further comprising obtaining the at least one related material.
9. The method of claim 1, further comprising obtaining the at least one related material via chemical transformation from at least one of the one or more putative base bioreachable molecules.
10. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set M4A Forward Synthesis: Chemically Transform BRMs to Other Molecules

1. A computer-implemented method for predicting properties of molecules derived from putative bioreachable molecules, the method comprising:
   a. transforming in silico a first putative bioreachable molecule of one or more putative bioreachable molecules to produce a second molecule; and
   b. predicting at least one property of the second molecule.
2. The method of claim 1, wherein predicting the at least one property is based at least in part upon chemical modeling of the first putative bioreachable molecule.
3. The method of any one of claim 1 or 2, wherein predicting the at least one property is based at least in part upon machine learning.
4. The method of claim 1, wherein transforming includes chemically transforming.
5. The method of claim 1, wherein transforming requires at most 2 reaction steps.
6. The method of claim 1, wherein transforming requires at most 3 reaction steps.
7. The method of claim 1, further comprising obtaining the second molecule.
8. The method of claim 1, further comprising obtaining the second molecule via transformation of the first putative bioreachable molecule.
9. The method of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Systems

### Set S1A Predicting Properties of Bioreachable Molecules

1. A system for predicting at least one property of a first molecule of one or more putative bioreachable molecules, the system comprising:
   one or more processors; and
   one or more memories storing instructions, that when executed by at least one of the one or more processors, cause the system to:
   generate a chemical model of the first molecule based on physicochemical properties; and
   predict the at least one property of the first molecule based at least in part upon the chemical model and empirical data concerning the first molecule, wherein the empirical data does not include data concerning the at least one property.
2. The system of claim 1, wherein predicting the at least one property of the first molecule employs statistical modeling or machine learning.
3. The system of claim 1, wherein at least one of the one or more memories stores instructions, that when executed by at least one of the one or more processors, cause the system to obtain the first molecule.
4. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set S1B Query Database to Identify BRM with Desired BRM Properties
1. A system for identifying a putative bioreachable molecule having a desired property, the system comprising:
   one or more processors; and
   one or more memories storing instructions, that when executed by at least one of the one or more processors, cause the system to:
      a. receive a query of a database, the query indicating a desired property, wherein:
         i. the database stores associations between one or putative bioreachable molecules and one or more predicted properties of the one or more putative bioreachable molecules, and
         iii. the one or more predicted properties include the desired property; and
      b. return data representing a first putative bioreachable molecule of the one or more putative bioreachable molecules based at least in part upon association of the first putative bioreachable molecule with the desired property.
2. The system of claim 1, wherein the one or more predicted properties are based at least in part upon chemical modeling of the one or more putative bioreachable molecules.
3. The system of any one of claim 1 or 2, wherein the one or more predicted properties are based at least in part upon empirical data concerning the one or more putative bioreachable molecules.
4. The system of claim 1, wherein the one or more predicted properties are based at least in part upon statistical modeling or machine learning.
5. The system of claim 1, wherein at least one of the one or more memories stores instructions that, when executed by at least one of the one or more processors, cause the system to obtain the first putative bioreachable molecule.
6. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set S2A Predict Material Properties Based Upon Related Bioreachable Molecule

1. A system for predicting at least one property of a material related to a first molecule of one or more putative bioreachable molecules, the system comprising:
   one or more processors; and
   one or more memories storing instructions, that when executed by at least one of the one or more processors, cause the system to:
      a. generate a chemical model of the material based on physicochemical properties; and
      b. predict the at least one property of the material based at least in part upon the chemical model and correlative modeling.
2. The system of claim 1, wherein predicting the at least one property of the material is based at least in part upon predictive modeling of the first molecule.
3. The system of claim 1, wherein the material comprises at least the first molecule.
4. The system of claim 1, wherein the material comprises at least two instances of the first molecule in its structure.
5. The system of claim 1, wherein the material comprises in its chemical structure at least the first molecule or at least one semi-synthetic molecule derived from the first molecule, or a combination thereof.
6. The system of claim 1, wherein at least one of the one or more memories stores instructions, that when executed by at least one of the one or more processors, cause the system to obtain the material.
7. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set S2B Query Database to Identify a Material with Desired Material Property
1. A system for identifying a material having a desired material property, wherein the material is related to one or more putative bioreachable molecules, the system comprising:
   one or more processors; and
   one or more memories storing instructions, that when executed by at least one of the one or more processors, cause the system to:
      a. receive a query of a database, the query indicating a desired material property, wherein:
         the database stores associations between one or more materials and one or more predicted material properties of the one or more materials, and
         the one or more predicted material properties include the desired material property,
         the one or more predicted material properties are based at least in part upon chemical modeling of the one or more putative bioreachable molecules; and
      b. return data representing a first material of the one or more materials based at least in part upon association of the first material with the desired material property.
6. The system of claim 1, wherein the associations are based at least in part upon statistical modeling or machine learning.
7. The system of claim 1, wherein the first material comprises in its chemical structure at least one bioreachable molecule of the one or more putative bioreachable molecules or at least one semi-synthetic molecule, or a combination thereof.
8. The system of claim 1, wherein at least one of the one or more memories stores instructions, that when executed by at least one of the one or more processors, cause the system to obtain the first material.
9. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set S3A Query DB to Identify BRM that can be Used to Produce Material with Desired Material Property
1. A system for identifying one or more putative bioreachable molecules related to a desired material property, the system comprising:
   one or more processors; and
   one or more memories storing instructions, that when executed by at least one of the one or more processors, cause the system to:
      a. receive a query of a database, the query indicating a desired material property, wherein the database stores associations between one or more putative bioreachable molecules and material properties of one or more materials; and
      b. return data representing one or more putative base bioreachable molecules of the one or more putative bioreachable molecules,
         i. wherein the one or more putative base bioreachable molecules are related to at least one material, of the one or more materials, that has the desired material property.
2. The system of claim 1, wherein the at least one related material includes within its structure the one or more putative base bioreachable molecules.
3. The system of claim 1, wherein the at least one related material is a polymer and each putative base bioreachable molecule of the one or more putative base bioreachable molecules is a monomer.
4. The system of claim 1, wherein the at least one related material comprises in its chemical structure at least one base bioreachable molecule of the one or more putative base bioreachable molecules or at least one semi-synthetic molecule, or a combination thereof.
5. The system of claim 1, wherein the association between the one or more putative base bioreachable molecules and the desired material property is based at least in part upon predictive modeling.
6. The system of claim 6, wherein the predictive modeling employs statistical modeling or machine learning.
7. The system of claim 1, wherein the returned data includes data representing at least one reaction pathway between the one or more putative base bioreachable molecules and the at least one related material.
8. The system of claim 1, wherein at least one of the one or more memories stores instructions, that when executed by at least one of the one or more processors, cause the system to obtain the at least one related material.
9. The system of claim 1, wherein at least one of the one or more memories stores instructions, that when executed by at least one of the one or more processors, cause the system to obtain the at least one related material via chemical transformation from at least one of the one or more putative base bioreachable molecules.
10. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set S4A Forward Synthesis: Chemically Transform BRMs to Other Molecules

1. A system for predicting properties of molecules derived from putative bioreachable molecules, the system comprising:
   one or more processors; and
   one or more memories storing instructions, that when executed by at least one of the one or more processors, cause the system to:
      a. transform in silico a first putative bioreachable molecule of one or more putative bioreachable molecules to produce a second molecule; and
      b. predict at least one property of the second molecule.
2. The system of claim 1, wherein predicting the at least one property is based at least in part upon chemical modeling of the first putative bioreachable molecule.
3. The system of any one of claim 1 or 2, wherein predicting the at least one property is based at least in part upon machine learning.
4. The system of claim 1, wherein transforming includes chemically transforming.
5. The system of claim 1, wherein transforming requires at most 2 reaction steps.
6. The system of claim 1, wherein transforming requires at most 3 reaction steps.
7. The system of claim 1, further comprising obtaining the second molecule.
8. The system of claim 1, further comprising obtaining the second molecule via transformation of the first putative bioreachable molecule.
9. The system of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Computer-Readable Media

### Set C1A Predicting Properties of Bioreachable Molecules

1. One or more non-transitory computer-readable media storing instructions for predicting at least one property of a first molecule of one or more putative bioreachable molecules, wherein the instructions, when executed by one or more computing devices, cause at least one of the one or more computing devices to:
   generate a chemical model of the first molecule based on physicochemical properties; and
   predict the at least one property of the first molecule based at least in part upon the chemical model and empirical data concerning the first molecule, wherein the empirical data does not include data concerning the at least one property.
2. The one or more non-transitory computer-readable media of claim 1, wherein predicting the at least one property of the first molecule employs statistical modeling or machine learning.
3. The one or more non-transitory computer-readable media of claim 1, wherein the instructions, when executed, cause at least one of the one or more computing devices to obtain the first molecule.
4. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set C1B Query Database to Identify BRM with Desired BRM Properties
1. One or more non-transitory computer-readable media storing instructions for identifying a putative bioreachable molecule having a desired property, wherein the instructions, when executed by one or more computing devices, cause at least one of the one or more computing devices to:
   a. receive a query of a database, the query indicating a desired property, wherein:
      i. the database stores associations between one or putative bioreachable molecules and one or more predicted properties of the one or more putative bioreachable molecules, and
      iv. the one or more predicted properties include the desired property; and
   b. return data representing a first putative bioreachable molecule of the one or more putative bioreachable molecules based at least in part upon association of the first putative bioreachable molecule with the desired property.
2. The one or more non-transitory computer-readable media of claim 1, wherein the one or more predicted properties are based at least in part upon chemical modeling of the one or more putative bioreachable molecules.
3. The one or more non-transitory computer-readable media of any one of claim 1 or 2, wherein the one or more predicted properties are based at least in part upon empirical data concerning the one or more putative bioreachable molecules.
4. The one or more non-transitory computer-readable media of claim 1, wherein the one or more predicted properties are based at least in part upon statistical modeling or machine learning.
5. The one or more non-transitory computer-readable media of claim 1, wherein the instructions, when executed, cause at least one of the one or more computing devices to obtain the first putative bioreachable molecule.
6. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set C2A Predict Material Properties Based Upon Related Bioreachable Molecule

1. One or more non-transitory computer-readable media storing instructions for predicting at least one property of a material related to a first molecule of one or more putative bioreachable molecules, wherein the instructions, when executed by one or more computing devices, cause at least one of the one or more computing devices to:
   a. generate a chemical model of the material based on physicochemical properties; and
   b. predict the at least one property of the material based at least in part upon the chemical model and correlative modeling.
2. The one or more non-transitory computer-readable media of claim 1, wherein predicting the at least one property of the material is based at least in part upon predictive modeling of the first molecule.
3. The one or more non-transitory computer-readable media of claim 1, wherein the material comprises at least the first molecule.
4. The one or more non-transitory computer-readable media of claim 1, wherein the material comprises at least two instances of the first molecule in its structure.
5. The one or more non-transitory computer-readable media of claim 1, wherein the material comprises in its chemical structure at least the first molecule or at least one semi-synthetic molecule derived from the first molecule, or a combination thereof.
6. The one or more non-transitory computer-readable media of claim 1, wherein the instructions, when executed, cause at least one of the one or more computing devices to obtain the material.
7. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set C2B Query database to identify a material with desired material property
1. One or more non-transitory computer-readable media storing instructions for identifying a material having a desired material property, wherein the material is related to one or more putative bioreachable molecules wherein the instructions, when executed by one or more computing devices, cause at least one of the one or more computing devices to:
   a. receive a query of a database, the query indicating a desired material property, wherein:
      the database stores associations between one or more materials and one or more predicted material properties of the one or more materials, and
      the one or more predicted material properties include the desired material property,
      the one or more predicted material properties are based at least in part upon chemical modeling of the one or more putative bioreachable molecules; and
   b. return data representing a first material of the one or more materials based at least in part upon association of the first material with the desired material property.
2. The one or more non-transitory computer-readable media of claim 1, wherein the associations are based at least in part upon statistical modeling or machine learning.
3. The one or more non-transitory computer-readable media of claim 1, wherein the first material comprises in its chemical structure at least one bioreachable molecule of the one or more putative bioreachable molecules or at least one semi-synthetic molecule, or a combination thereof.
4. The one or more non-transitory computer-readable media of claim 1, wherein the instructions, when executed, cause at least one of the one or more computing devices to obtain the first material.
5. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.
      Set C3A Query DB to Identify BRM that can be Used to Produce Material with Desired Material Property
1. One or more non-transitory computer-readable media storing instructions for identifying one or more putative bioreachable molecules related to a desired material property, wherein the instructions, when executed by one or more computing devices, cause at least one of the one or more computing devices to:
   a. receive a query of a database, the query indicating a desired material property, wherein the database stores associations between one or more putative bioreachable molecules and material properties of one or more materials; and
   b. return data representing one or more putative base bioreachable molecules of the one or more putative bioreachable molecules,
      i. wherein the one or more putative base bioreachable molecules are related to at least one material, of the one or more materials, that has the desired material property.
2. The one or more non-transitory computer-readable media of claim 1, wherein the at least one related material includes within its structure the one or more putative base bioreachable molecules.
3. The one or more non-transitory computer-readable media of claim 1, wherein the at least one related material is a polymer and each putative base bioreachable molecule of the one or more putative base bioreachable molecules is a monomer.
4. The one or more non-transitory computer-readable media of claim 1, wherein the at least one related material comprises in its chemical structure at least one base bioreachable molecule of the one or more putative base bioreachable molecules or at least one semi-synthetic molecule, or a combination thereof.
5. The one or more non-transitory computer-readable media of claim 1, wherein the association between the one or more putative base bioreachable molecules and the desired material property is based at least in part upon predictive modeling.
6. The one or more non-transitory computer-readable media of claim 6, wherein the predictive modeling employs statistical modeling or machine learning.
7. The one or more non-transitory computer-readable media of claim 1, wherein the returned data includes data representing at least one reaction pathway between the one or more putative base bioreachable molecules and the at least one related material.
8. The one or more non-transitory computer-readable media of claim 1, wherein the instructions, when executed, cause at least one of the one or more computing devices to obtain the at least one related material.
9. The one or more non-transitory computer-readable media of claim 1, wherein the instructions, when executed, cause at least one of the one or more computing devices to obtain the at least one related material via chemical transformation from at least one of the one or more putative base bioreachable molecules.
10. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Set C4A Forward Synthesis: Chemically Transform BRMs to Other Molecules

1. One or more non-transitory computer-readable media storing instructions for predicting properties of molecules derived from putative bioreachable molecules, wherein the instructions, when executed by one or more computing devices, cause at least one of the one or more computing devices to:
   a. transform in silico a first putative bioreachable molecule of one or more putative bioreachable molecules to produce a second molecule; and
   b. predict at least one property of the second molecule.
2. The one or more non-transitory computer-readable media of claim 1, wherein predicting the at least one property is based at least in part upon chemical modeling of the first putative bioreachable molecule.
3. The one or more non-transitory computer-readable media of any one of claim 1 or 2, wherein predicting the at least one property is based at least in part upon machine learning.
4. The one or more non-transitory computer-readable media of claim 1, wherein transforming includes chemically transforming.
5. The one or more non-transitory computer-readable media of claim 1, wherein transforming requires at most 2 reaction steps.
6. The one or more non-transitory computer-readable media of claim 1, wherein transforming requires at most 3 reaction steps.
7. The one or more non-transitory computer-readable media of claim 1, further comprising obtaining the second molecule.
8. The one or more non-transitory computer-readable media of claim 1, further comprising obtaining the second molecule via transformation of the first putative bioreachable molecule.
9. The one or more non-transitory computer-readable media of claim 1, wherein at least one of the one or more putative bioreachable molecules is determined by:
   obtaining, using at least one processor, a starting metabolite set specifying starting metabolites for a host organism;
   obtaining, using at least one processor, a starting reaction set specifying reactions;
   using at least one processor, including in a filtered reaction set one or more reactions from the starting reaction set; and
   in each processing step of one or more processing steps performed by at least one processor, processing, pursuant to the one or more reactions of the filtered reaction set, data representing the starting metabolites and metabolites generated in previous processing steps, to generate data representing the at least one putative bioreachable molecule.

### Further Embodiments

1. A computer-implemented method for predicting at least one property of a first molecule of one or more putative bioreachable molecules, the method comprising:

accessing a predictive model of the first molecule that employs (a) statistical modeling or machine learning or (b) chemical modeling;

predicting at least one property of the first molecule based at least in part upon the predictive model; and

returning data representing the at least one property.
2. The method of claim 1, wherein predicting the at least one property of the first molecule is based at least in part upon prediction using (a) statistical modeling or machine learning and (b) chemical modeling.
3. The method of claim 1, wherein predicting the at least one property of the first molecule comprises prediction using statistical modeling or machine learning, and then prediction using chemical modeling.
4. The method of claim 1, further comprising obtaining the first molecule.
5. A system for predicting at least one property of a first molecule of one or more putative bioreachable molecules, the system comprising:

one or more processors; and

one or more memories operatively coupled to the one or more processors and storing instructions, that when executed by at least one of the one or more processors, cause the system to:

access a predictive model of the first molecule that employs (a) statistical modeling or machine learning or (b) chemical modeling;

predict at least one property of the first molecule based at least in part upon the predictive model; and

return data representing the at least one property.
6. The system of claim 5, wherein predicting the at least one property of the first molecule is based at least in part upon prediction using (a) statistical modeling or machine learning and (b) chemical modeling.
7. The system of claim 5, wherein predicting the at least one property of the first molecule comprises prediction using statistical modeling or machine learning, and then prediction using chemical modeling.
8. The system of claim 5, the one or more memories storing instructions that when executed cause the first molecule to be obtained.
9. One or more non-transitory computer-readable media storing instructions for predicting at least one property of a first molecule of one or more putative bioreachable molecules, wherein the instructions, when executed by one or more computing devices, cause at least one of the one or more computing devices to:

access a predictive model of the first molecule that employs (a) statistical modeling or machine learning or (b) chemical modeling;

predict at least one property of the first molecule based at least in part upon the predictive model; and

return data representing the at least one property.
10. The one or more non-transitory computer-readable media of claim 9, wherein predicting the at least one property of the first molecule is based at least in part upon prediction using (a) statistical modeling or machine learning and (b) chemical modeling.
11. The one or more non-transitory computer-readable media of claim 9, wherein predicting the at least one property of the first molecule comprises prediction using statistical modeling or machine learning, and then prediction using chemical modeling.
12. The one or more non-transitory computer-readable media of claim 9, storing instructions that, when executed, cause the first molecule to be obtained.
13. A computer-implemented method for identifying a set of putative bioreachable molecules having one or more desired properties, the method comprising:

receiving one or more queries indicating one or more desired properties;

determining data representing a set of putative bioreachable molecules based at least in part upon association of the set of putative bioreachable molecules with the one or more desired properties, wherein at least one of the one or more desired properties is based at least in part upon prediction using (a) statistical modeling or machine learning or (b) chemical modeling; and

returning data representing the determined set.
14. The method of claim 13, wherein at least one of the one or more desired properties is based at least in part upon prediction using statistical modeling or machine learning and at least one other of the desired properties is based at least in part upon prediction using chemical modeling.
15. The method of claim 13, wherein the at least one of the one or more desired properties is based at least in part upon prediction using (a) statistical modeling or machine learning and (b) chemical modeling.
16. The method of claim 13, wherein at least one of the one or more desired properties is initially based at least in part upon prediction using statistical modeling or machine learning, and the determined set comprises a reduced number of putative bioreachable molecules after later predicting at least one of the one or more desired properties using chemical modeling.
17. The method of claim 13, wherein determining comprises determining data representing a set of putative bioreachable molecules based at least in part upon (a) association of the set of putative bioreachable molecules with the one or more desired properties and (b) a maximum distance between (i) one or more putative bioreachable molecules within the set and (ii) an ancestor putative bioreachable molecule satisfying a desired maximum distance.
18. The method of claim 13, further comprising obtaining at least one putative bioreachable molecule of the set.
19. A system for identifying a set of putative bioreachable molecules having one or more desired properties, the system comprising:

one or more processors; and

one or more memories operatively coupled to the one or more processors and storing instructions, that when executed by at least one of the one or more processors, cause the system to:

receive one or more queries indicating one or more desired properties;

determine data representing a set of putative bioreachable molecules based at least in part upon association of the set of putative bioreachable molecules with the one or more desired properties, wherein at least one of the one or more desired properties is based at least in part upon prediction using (a) statistical modeling or machine learning or (b) chemical modeling; and

return data representing the determined set.
20. The system of claim 19, wherein at least one of the one or more desired properties is based at least in part upon prediction using statistical modeling or machine learning and at least one other of the desired properties is based at least in part upon prediction using chemical modeling.
21. The system of claim 19, wherein the at least one of the one or more desired properties is based at least in part upon prediction using (a) statistical modeling or machine learning and (b) chemical modeling.
22. The system of claim 19, wherein at least one of the one or more desired properties is initially based at least in part upon prediction using statistical modeling or machine learning, and the determined set comprises a reduced number of putative bioreachable molecules after later predicting at least one of the one or more desired properties using chemical modeling.
23. The system of claim 19, wherein determining comprises determining data representing a set of putative bioreachable molecules based at least in part upon (a) association of the set of putative bioreachable molecules with the one or more desired properties and (b) a maximum distance between (i) one or more putative bioreachable molecules within the set and (ii) an ancestor putative bioreachable molecule satisfying a desired maximum distance.
24. The system of claim 19, the one or more memories storing instructions that when executed cause the first molecule to be obtained.
25. One or more non-transitory computer-readable media storing instructions for identifying a set of putative bioreachable molecules having one or more desired properties, wherein the instructions, when executed by one or more computing devices, cause at least one of the one or more computing devices to:

receive one or more queries indicating one or more desired properties;

determine data representing a set of putative bioreachable molecules based at least in part upon association of the set of putative bioreachable molecules with the one or more desired properties, wherein at least one of the one or more desired properties is based at least in part upon prediction using (a) statistical modeling or machine learning or (b) chemical modeling; and

return data representing the determined set.
26. The one or more non-transitory computer-readable media of claim 25, wherein at least one of the one or more desired properties is based at least in part upon prediction using statistical modeling or machine learning and at least one other of the desired properties is based at least in part upon prediction using chemical modeling.
27. The one or more non-transitory computer-readable media of claim 25, wherein the at least one of the one or more desired properties is based at least in part upon prediction using (a) statistical modeling or machine learning and (b) chemical modeling.
28. The one or more non-transitory computer-readable media of claim 25, wherein at least one of the one or more desired properties is initially based at least in part upon prediction using statistical modeling or machine learning, and the determined set comprises a reduced number of putative bioreachable molecules after later predicting at least one of the one or more desired properties using chemical modeling.
29. The one or more non-transitory computer-readable media of claim 25, wherein determining comprises determining data representing a set of putative bioreachable molecules based at least in part upon (a) association of the set of putative bioreachable molecules with the one or more desired properties and (b) a maximum distance between (i) one or more putative bioreachable molecules within the set and (ii) an ancestor putative bioreachable molecule satisfying a desired maximum distance.
30. The one or more non-transitory computer-readable media of claim 25, storing instructions that, when executed, cause the first molecule to be obtained.
31. A computer-implemented method for predicting at least one property of a material related to a first molecule of one or more putative bioreachable molecules, the method comprising:

accessing a predictive model that employs (a) chemical modeling or (b) statistical modeling or machine learning;

predicting at least one property of the material based at least in part upon the predictive model; and

returning data representing the at least one property.
32. The method of claim 31, wherein predicting the at least one property of the material is based at least in part upon prediction using (a) chemical modeling and (b) statistical modeling or machine learning.
33. The method of claim 31, wherein predicting the at least one property of the material comprises prediction using statistical modeling or machine learning, and then prediction using chemical modeling.
34. The method of claim 31, wherein the material comprises at least the first molecule.
35. The method of claim 31, wherein the material comprises at least two instances of the first molecule in its structure.
36. The method of claim 31, wherein the material comprises in its chemical structure at least the first molecule or at least one semi-synthetic molecule derived from the first molecule, or a combination thereof.
37. The method of claim 31, further comprising obtaining the material.
38. A system for predicting at least one property of a material related to a first molecule of one or more putative bioreachable molecules, the system comprising:

one or more processors; and

one or more memories operatively coupled to the one or more processors and storing instructions, that when executed by at least one of the one or more processors, cause the system to:

access a predictive model that employs (a) chemical modeling or (b) statistical modeling or machine learning;

predict at least one property of the material based at least in part upon the predictive model; and

return data representing the at least one property.
39. The system of claim 38, wherein predicting the at least one property of the material is based at least in part upon prediction using (a) chemical modeling and (b) statistical modeling or machine learning.
40. The system of claim 38, wherein predicting the at least one property of the material comprises prediction using statistical modeling or machine learning, and then prediction using chemical modeling.
41. The system of claim 38, wherein the material comprises at least the first molecule.
42. The system of claim 38, wherein the material comprises at least two instances of the first molecule in its structure.
43. The system of claim 38, wherein the material comprises in its chemical structure at least the first molecule or at least one semi-synthetic molecule derived from the first molecule, or a combination thereof.
44. The system of claim 38, the one or more memories storing instructions that when executed cause the first molecule to be obtained.
45. One or more non-transitory computer-readable media storing instructions for predicting at least one property of a material related to a first molecule of one or more putative bioreachable molecules, wherein the instructions, when executed by one or more computing devices, cause at least one of the one or more computing devices to:

access a predictive model that employs (a) chemical modeling or (b) statistical modeling or machine learning;

predict at least one property of the material based at least in part upon the predictive model; and

return data representing the at least one property.
46. The one or more non-transitory computer-readable media of claim 45, wherein predicting the at least one property of the material is based at least in part upon prediction using (a) chemical modeling and (b) statistical modeling or machine learning.
47. The one or more non-transitory computer-readable media of claim 45, wherein predicting the at least one property of the material comprises prediction using statistical modeling or machine learning, and then prediction using chemical modeling.
48. The one or more non-transitory computer-readable media of claim 45, wherein the material comprises at least the first molecule.
49. The one or more non-transitory computer-readable media of claim 45, wherein the material comprises at least two instances of the first molecule in its structure.
50. The one or more non-transitory computer-readable media of claim 45, wherein the material comprises in its chemical structure at least the first molecule or at least one semi-synthetic molecule derived from the first molecule, or a combination thereof.
51. The one or more non-transitory computer-readable media of claim 45 storing instructions that when executed cause the first molecule to be obtained.
52. A computer-implemented method for identifying a set of materials having one or more desired material properties, the method comprising:

receiving one or more queries indicating one or more desired material properties;

determining data representing a set of materials based at least in part upon association of the set of materials with the one or more desired material properties, wherein the set of materials is related to one or more putative bioreachable molecules, and at least one of the one or more desired material properties is based at least in part upon prediction using (a) chemical modeling or (b) statistical modeling or machine learning; and

returning data representing the determined set.
53. The method of claim 52, wherein at least one of the one or more desired material properties is based at least in part upon prediction using chemical modeling and at least one other of the desired material properties is based at least in part upon prediction using statistical modeling or machine learning.
54. The method of claim 52, wherein the at least one of the one or more desired material properties is based at least in part upon prediction using (a) chemical modeling and (b) statistical modeling or machine learning.
55. The method of claim 52, wherein at least one of the one or more desired material properties is initially based at least in part upon prediction using statistical modeling or machine learning, and the determined set comprises a reduced number of materials after later predicting at least one of the one or more desired material properties using chemical modeling.
56. The method of claim 52, wherein determining comprises determining data representing a set of materials based at least in part upon (a) association of the set of materials with the one or more desired material properties and (b) a maximum distance between (i) one or more putative bioreachable molecules related to the set and (ii) an ancestor putative bioreachable molecule satisfying a desired maximum distance.
57. The method of claim 52, wherein the set of materials comprises one or more materials that each comprise in its chemical structure at least one of the one or more putative bioreachable molecules, or at least one semi-synthetic molecule related to at least one of the one or more putative bioreachable molecules, or a combination thereof.
58. The method of claim 52, further comprising obtaining at least one material of the set of materials or at least one of the one or more putative bioreachable molecules.
59. A system for identifying a set of materials having one or more desired material properties, the system comprising:

one or more processors; and

one or more memories operatively coupled to the one or more processors and storing instructions, that when executed by at least one of the one or more processors, cause the system to:

receive one or more queries indicating one or more desired material properties;

determine data representing a set of materials based at least in part upon association of the set of materials with the one or more desired material properties, wherein the set of materials is related to one or more putative bioreachable molecules, and at least one of the one or more desired material properties is based at least in part upon prediction using (a) chemical modeling or (b) statistical modeling or machine learning; and

return data representing the determined set.
60. The system of claim 59, wherein at least one of the one or more desired material properties is based at least in part upon prediction using chemical modeling and at least one other of the desired material properties is based at least in part upon prediction using statistical modeling or machine learning.
61. The system of claim 59, wherein the at least one of the one or more desired material properties is based at least in part upon prediction using (a) chemical modeling and (b) statistical modeling or machine learning.
62. The system of claim 59, wherein at least one of the one or more desired material properties is initially based at least in part upon prediction using statistical modeling or machine learning, and the determined set comprises a reduced number of materials after later predicting at least one of the one or more desired material properties using chemical modeling.
63. The system of claim 59, wherein determining comprises determining data representing a set of materials based at least in part upon (a) association of the set of materials with the one or more desired material properties and (b) a maximum distance between (i) one or more putative bioreachable molecules related to the set and (ii) an ancestor putative bioreachable molecule satisfying a desired maximum distance.
64. The system of claim 59, wherein the set of materials comprises one or more materials that each comprise in its chemical structure at least one of the one or more putative bioreachable molecules, or at least one semi-synthetic molecule related to at least one of the one or more putative bioreachable molecules, or a combination thereof.
65. The system of claim 59, the one or more memories storing instructions that when executed cause at least one material of the set of materials or at least one of the one or more putative bioreachable molecules to be obtained.
66. One or more non-transitory computer-readable media storing instructions for identifying one or more materials having one or more desired material properties, wherein the instructions, when executed by one or more computing devices, cause at least one of the one or more computing devices to:

receive one or more queries indicating one or more desired material properties;

determine data representing a set of materials based at least in part upon association of the set of materials with the one or more desired material properties, wherein the set of materials is related to one or more putative bioreachable molecules, and at least one of the one or more desired material properties is based at least in part upon prediction using (a) chemical modeling or (b) statistical modeling or machine learning; and

return data representing the determined set.
67. The one or more non-transitory computer-readable media of claim 66, wherein at least one of the one or more desired material properties is based at least in part upon prediction using chemical modeling and at least one other of the desired material properties is based at least in part upon prediction using statistical modeling or machine learning.
68. The one or more non-transitory computer-readable media of claim 66, wherein the at least one of the one or more desired material properties is based at least in part upon prediction using (a) chemical modeling and (b) statistical modeling or machine learning.
69. The one or more non-transitory computer-readable media of claim 66, wherein at least one of the one or more desired material properties is initially based at least in part upon prediction using statistical modeling or machine learning, and the determined set comprises a reduced number of materials after later predicting at least one of the one or more desired material properties using chemical modeling.
70. The one or more non-transitory computer-readable media of claim 66, wherein determining comprises determining data representing a set of materials based at least in part upon (a) association of the set of materials with the one or more desired material properties and (b) a maximum distance between (i) one or more putative bioreachable molecules related to the set and (ii) an ancestor putative bioreachable molecule satisfying a desired maximum distance.
71. The one or more non-transitory computer-readable media of claim 66, wherein the set of materials comprises one or more materials that each comprise in its chemical structure at least one of the one or more putative bioreachable molecules, or at least one semi-synthetic molecule related to at least one of the one or more putative bioreachable molecules, or a combination thereof.
72. The one or more non-transitory computer-readable media of claim 66 storing instructions that when executed cause at least one material of the set of materials or at least one of the one or more putative bioreachable molecules to be obtained.
73. A computer-implemented method for identifying one or more putative bioreachable molecules related to one or more desired material properties, the method comprising:

receiving one or more queries indicating one or more desired material properties; and

determining data representing a set of putative bioreachable molecules based at least in part upon association of the set of putative bioreachable molecules with the one or more desired material properties, wherein the set of putative bioreachable molecules is related to one or more materials, and at least one of the one or more desired material properties is based at least in part upon prediction using (a) chemical modeling or (b) statistical modeling or machine learning; and

returning data representing the determined set.
74. The method of claim 73, wherein at least one of the one or more desired material properties is based at least in part upon prediction using chemical modeling and at least one other of the desired material properties is based at least in part upon prediction using statistical modeling or machine learning.
75. The method of claim 73, wherein the at least one of the one or more desired material properties is based at least in part upon prediction using (a) chemical and (b) statistical modeling or machine learning.
76. The method of claim 73, wherein at least one of the one or more desired material properties is initially based at least in part upon prediction using statistical modeling or machine learning, and the determined set comprises a reduced number of putative bioreachable molecules after later predicting at least one of the one or more desired material properties using chemical modeling.
77. The method of claim 73, wherein determining comprises determining data representing a set of putative bioreachable molecules based at least in part upon (a) association of the set of putative bioreachable molecules with the one or more desired material properties and (b) a maximum distance between (i) one or more putative bioreachable molecules of the set and (ii) an ancestor putative bioreachable molecule satisfying a desired maximum distance.
78. The method of claim 73, wherein the one or more related materials each includes within its chemical structure at least one putative bioreachable molecule of the set, at least one semi-synthetic molecule, or a combination thereof.
79. The method of claim 73, wherein the returned data includes data representing at least one reaction pathway between at least one putative bioreachable molecule of the set and at least one related material.
80. The method of claim 73, further comprising obtaining at least one of the one or more materials or at least one putative bioreachable molecule of the set.
81. The method of claim 73, further comprising obtaining at least one of the one or more materials via chemical transformation from at least one putative bioreachable molecule of the set.
82. A system for identifying one or more putative bioreachable molecules related to one or more desired material properties, the system comprising:

one or more processors; and

one or more memories operatively coupled to the one or more processors and storing instructions, that when executed by at least one of the one or more processors, cause the system to:

receive one or more queries indicating one or more desired material properties; and

determine data representing a set of putative bioreachable molecules based at least in part upon association of the set of putative bioreachable molecules with the one or more desired material properties, wherein the set of putative bioreachable molecules is related to one or more materials, and at least one of the one or more desired material properties is based at least in part upon prediction using (a) chemical or (b) statistical modeling or machine learning; and

return data representing the determined set.
83. The system of claim 82, wherein at least one of the one or more desired material properties is based at least in part upon prediction using chemical modeling and at least one other of the desired material properties is based at least in part upon prediction using statistical modeling or machine learning.
84. The system of claim 82, wherein the at least one of the one or more desired material properties is based at least in part upon prediction using (a) chemical modeling and (b) statistical modeling or machine learning.
85. The system of claim 82, wherein at least one of the one or more desired material properties is initially based at least in part upon prediction using statistical modeling or machine learning, and the determined set comprises a reduced number of putative bioreachable molecules after later predicting at least one of the one or more desired material properties using chemical modeling.
86. The system of claim 82, wherein determining comprises determining data representing a set of putative bioreachable molecules based at least in part upon (a) association of the set of putative bioreachable molecules with the one or more desired material properties and (b) a maximum distance between (i) one or more putative bioreachable molecules of the set and (ii) an ancestor putative bioreachable molecule satisfying a desired maximum distance.
87. The system of claim 82, wherein the one or more related materials each includes within its chemical structure at least one putative bioreachable molecule of the set, at least one semi-synthetic molecule, or a combination thereof.
88. The system of claim 82, wherein the returned data includes data representing at least one reaction pathway between at least one putative bioreachable molecule of the set and at least one related material.
89. The system of claim 82, the one or more memories storing instructions that when executed cause at least one of the one or more materials or at least one putative bioreachable molecule of the set to be obtained.
90. The system of claim 82, the one or more memories storing instructions that when executed cause at least one of the one or more materials to be obtained via chemical transformation from at least one putative bioreachable molecule of the set.
91. One or more non-transitory computer-readable media storing instructions for identifying one or more putative bioreachable molecules related to one or more desired material properties, wherein the instructions, when executed by one or more computing devices, cause at least one of the one or more computing devices to:

receive one or more queries indicating one or more desired material properties; and

determine data representing a set of putative bioreachable molecules based at least in part upon association of the set of putative bioreachable molecules with the one or more desired material properties, wherein the set of putative bioreachable molecules is related to one or more materials, and at least one of the one or more desired material properties is based at least in part upon prediction using (a) chemical modeling or (b) statistical modeling or machine learning; and

return data representing the determined set.
92. The one or more non-transitory computer-readable media of claim 91, wherein at least one of the one or more desired material properties is based at least in part upon prediction using chemical modeling and at least one other of the desired material properties is based at least in part upon prediction using statistical modeling or machine learning.
93. The one or more non-transitory computer-readable media of claim 91, wherein the at least one of the one or more desired material properties is based at least in part upon prediction using (a) chemical modeling and (b) statistical modeling or machine learning.
94. The one or more non-transitory computer-readable media of claim 91, wherein at least one of the one or more desired material properties is initially based at least in part upon prediction using statistical modeling or machine learning, and the determined set comprises a reduced number of putative bioreachable molecules after later predicting at least one of the one or more desired material properties using chemical modeling.
95. The one or more non-transitory computer-readable media of claim 91, wherein determining comprises determining data representing a set of putative bioreachable molecules based at least in part upon (a) association of the set of putative bioreachable molecules with the one or more desired material properties and (b) a maximum distance between (i) one or more putative bioreachable molecules of the set and (ii) an ancestor putative bioreachable molecule satisfying a desired maximum distance.
96. The one or more non-transitory computer-readable media of claim 91, wherein the one or more related materials each includes within its chemical structure at least one putative bioreachable molecule of the set, at least one semi-synthetic molecule, or a combination thereof.
97. The one or more non-transitory computer-readable media of claim 91, wherein the returned data includes data representing at least one reaction pathway between at least one putative bioreachable molecule of the set and at least one related material.
98. The one or more non-transitory computer-readable media of claim 91 storing instructions that when executed cause at least one of the one or more materials or at least one putative bioreachable molecule of the set to be obtained.
99. The one or more non-transitory computer-readable media of claim 91 storing instructions that when executed cause at least one of the one or more materials to be obtained via chemical transformation from at least one putative bioreachable molecule of the set.
100. A computer-implemented method for predicting properties of molecules derived from putative bioreachable molecules, the method comprising:
a. transforming in silico a first putative bioreachable molecule of one or more putative bioreachable molecules to produce a second molecule in silico;
b. predicting at least one property of the second molecule based at least in part upon (a) chemical modeling applied to the second molecule or (b) statistical modeling or machine learning; and
c. returning data representing the at least one property.
101. The method of claim 100, wherein predicting the at least one property of the second molecule is based at least in part upon prediction using (a) statistical modeling or machine learning and (b) chemical modeling.
102. The method of claim 100, wherein predicting the at least one property of the second molecule comprises prediction using statistical modeling or machine learning, and then prediction using chemical modeling.
103. The method of claim 100, wherein transforming includes chemically transforming.
104. The method of claim 100, wherein transforming requires at most 2 reaction steps.
105. The method of claim 100, wherein transforming requires at most 3 reaction steps.
106. The method of claim 100, further comprising obtaining the second molecule.
107. The method of claim 100, further comprising obtaining the second molecule via transformation of the first putative bioreachable molecule.
108. A system for predicting properties of molecules derived from putative bioreachable molecules, the system comprising:
   one or more processors; and

one or more memories operatively coupled to the one or more processors and storing instructions, that when executed by at least one of the one or more processors, cause the system to:
a. transform in silico a first putative bioreachable molecule of one or more putative bioreachable molecules to produce a second molecule in silico;
b. predict at least one property of the second molecule based at least in part upon (a) chemical modeling applied to the second molecule or (b) statistical modeling or machine learning; and
c. return data representing the at least one property.
109. The system of claim 108, wherein predicting the at least one property of the second molecule is based at least in part upon prediction using (a) statistical modeling or machine learning and (b) chemical modeling.
110. The system of claim 108, wherein predicting the at least one property of the second molecule comprises prediction using statistical modeling or machine learning, and then prediction using chemical modeling.
111. The system of claim 108, wherein transforming includes chemically transforming.
112. The system of claim 108, wherein transforming requires at most 2 reaction steps.
113. The system of claim 108, wherein transforming requires at most 3 reaction steps.
114. The system of claim 108, the one or more memories storing instructions that when executed cause the second molecule to be obtained.
115. The system of claim 108, the one or more memories storing instructions that when executed cause the second molecule to be obtained via transformation of the first putative bioreachable molecule.
116. One or more non-transitory computer-readable media storing instructions for predicting properties of molecules derived from putative bioreachable molecules, wherein the instructions, when executed by one or more computing devices, cause at least one of the one or more computing devices to:
a. transform in silico a first putative bioreachable molecule of one or more putative bioreachable molecules to produce a second molecule in silico;
b. predict at least one property of the second molecule based at least in part upon (a) chemical modeling applied to the second molecule or (b) statistical modeling or machine learning; and
c. return data representing the at least one property.
117. The one or more non-transitory computer-readable media of claim 116, wherein predicting the at least one property of the second molecule is based at least in part upon prediction using (a) statistical modeling or machine learning and (b) chemical modeling.
118. The one or more non-transitory computer-readable media of claim 116, wherein predicting the at least one property of the second molecule comprises prediction using statistical modeling or machine learning, and then prediction using chemical modeling.
119. The one or more non-transitory computer-readable media of claim 116, wherein transforming includes chemically transforming.
120. The one or more non-transitory computer-readable media of claim 116, wherein transforming requires at most 2 reaction steps.
121. The one or more non-transitory computer-readable media of claim 116, wherein transforming requires at most 3 reaction steps.
122. The one or more non-transitory computer-readable media of claim 116 storing instructions that when executed cause the second molecule to be obtained.
123. The one or more non-transitory computer-readable media of claim 116 storing instructions that when executed cause the second molecule to be obtained via transformation of the first putative bioreachable molecule.
124. The one or more non-transitory computer-readable media of claim 9, wherein the one or more putative bioreachable molecules are determined by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites and metabolites generated in previous processing steps, to generate data representing the one or more putative bioreachable molecules.
125. The one or more non-transitory computer-readable media of claim 124, wherein selecting comprises selecting reactions that are indicated as catalyzed by one or more corresponding catalysts that are themselves indicated as able to be engineered into an organism or taken up from the growth medium in which an organism is grown.
126. The one or more non-transitory computer-readable media of claim 124, wherein selecting comprises selecting reactions that are indicated as catalyzed by one or more corresponding catalysts that are themselves indicated as corresponding to one or more amino acid sequences or one or more genetic sequences.
127. The one or more non-transitory computer-readable media of claim 124, wherein selecting comprises selecting reactions based at least in part upon whether the reactions are indicated in at least one database as catalyzed by one or more corresponding catalysts that are themselves indicated as available to catalyze the reactions.
128. The one or more non-transitory computer-readable media of claim 25, wherein the putative bioreachable molecules are determined by:

a. selecting reactions based at least in part upon whether the reactions are indicated as catalyzed by one or more corresponding catalysts that are themselves indicated as available to catalyze the reactions, wherein a reaction set comprises the selected reactions; and

b. in each processing step of one or more processing steps, processing, pursuant to the one or more reactions in the reaction set, data representing starting metabolites and metabolites generated in previous processing steps, to generate data representing the putative bioreachable molecules.
129. The one or more non-transitory computer-readable media of claim 128, wherein selecting comprises selecting reactions that are indicated as catalyzed by one or more corresponding catalysts that are themselves indicated as able to be engineered into an organism or taken up from the growth medium in which an organism is grown.
130. The one or more non-transitory computer-readable media of claim 128, wherein selecting comprises selecting reactions that are indicated as catalyzed by one or more corresponding catalysts that are themselves indicated as corresponding to one or more amino acid sequences or one or more genetic sequences.
131. The one or more non-transitory computer-readable media of claim 128, wherein selecting comprises selecting reactions based at least in part upon whether the reactions are indicated in at least one database as catalyzed by one or more corresponding catalysts that are themselves indicated as available to catalyze the reactions.
132. The one or more non-transitory computer-readable media of claim 45, wherein the one or more putative bioreachable molecules are determined by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites and metabolites generated in previous processing steps, to generate data representing the one or more putative bioreachable molecules.
133. The one or more non-transitory computer-readable media of claim 132, wherein selecting comprises selecting reactions that are indicated as catalyzed by one or more corresponding catalysts that are themselves indicated as able to be engineered into an organism or taken up from the growth medium in which an organism is grown.
134. The one or more non-transitory computer-readable media of claim 132, wherein selecting comprises selecting reactions that are indicated as catalyzed by one or more corresponding catalysts that are themselves indicated as corresponding to one or more amino acid sequences or one or more genetic sequences.
135. The one or more non-transitory computer-readable media of claim 132, wherein selecting comprises selecting reactions based at least in part upon whether the reactions are indicated in at least one database as catalyzed by one or more corresponding catalysts that are themselves indicated as available to catalyze the reactions.
136. The one or more non-transitory computer-readable media of claim 66, wherein the one or more putative bioreachable molecules are determined by:
c. selecting reactions based at least in part upon whether the reactions are indicated as catalyzed by one or more corresponding catalysts that are themselves indicated as available to catalyze the reactions, wherein a reaction set comprises the selected reactions; and
d. in each processing step of one or more processing steps, processing, pursuant to the one or more reactions in the reaction set, data representing starting metabolites and metabolites generated in previous processing steps, to generate data representing the one or more putative bioreachable molecules.
137. The one or more non-transitory computer-readable media of claim 136, wherein selecting comprises selecting reactions that are indicated as catalyzed by one or more corresponding catalysts that are themselves indicated as able to be engineered into an organism or taken up from the growth medium in which an organism is grown.
138. The one or more non-transitory computer-readable media of claim 136, wherein selecting comprises selecting reactions that are indicated as catalyzed by one or more corresponding catalysts that are themselves indicated as corresponding to one or more amino acid sequences or one or more genetic sequences.
139. The one or more non-transitory computer-readable media of claim 136, wherein selecting comprises selecting reactions based at least in part upon whether the reactions are indicated in at least one database as catalyzed by one or more corresponding catalysts that are themselves indicated as available to catalyze the reactions.
140. The one or more non-transitory computer-readable media of claim 91, wherein the one or more putative bioreachable molecules are determined by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites and metabolites generated in previous processing steps, to generate data representing the one or more putative bioreachable molecules.
141. The one or more non-transitory computer-readable media of claim 140, wherein selecting comprises selecting reactions that are indicated as catalyzed by one or more corresponding catalysts that are themselves indicated as able to be engineered into an organism or taken up from the growth medium in which an organism is grown.
142. The one or more non-transitory computer-readable media of claim 140, wherein selecting comprises selecting reactions that are indicated as catalyzed by one or more corresponding catalysts that are themselves indicated as corresponding to one or more amino acid sequences or one or more genetic sequences.
143. The one or more non-transitory computer-readable media of claim 140, wherein selecting comprises selecting reactions based at least in part upon whether the reactions are indicated in at least one database as catalyzed by one or more corresponding catalysts that are themselves indicated as available to catalyze the reactions.
144. The one or more non-transitory computer-readable media of claim 116, wherein the one or more putative bioreachable molecules are determined by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites and metabolites generated in previous processing steps, to generate data representing the one or more putative bioreachable molecules.
145. The one or more non-transitory computer-readable media of claim 144, wherein selecting comprises selecting reactions that are indicated as catalyzed by one or more corresponding catalysts that are themselves indicated as able to be engineered into an organism or taken up from the growth medium in which an organism is grown.
146. The one or more non-transitory computer-readable media of claim 144, wherein selecting comprises selecting reactions that are indicated as catalyzed by one or more corresponding catalysts that are themselves indicated as corresponding to one or more amino acid sequences or one or more genetic sequences.
147. The one or more non-transitory computer-readable media of claim 144, wherein selecting comprises selecting reactions based at least in part upon whether the reactions are indicated in at least one database as catalyzed by one or more corresponding catalysts that are themselves indicated as available to catalyze the reactions.

End of Embodiments Section

What is claimed is:

1. One or more non-transitory computer-readable media storing instructions for predicting at least one property of a first molecule of one or more putative bioreachable molecules, wherein the instructions, when executed by one or more computing devices, cause at least one of the one or more computing devices to:
access a predictive model of the first molecule that employs (a) statistical modeling or machine learning or (b) chemical modeling;
predict at least one property of the first molecule based at least in part upon the predictive model; and
return data representing the at least one property.

2. The one or more non-transitory computer-readable media of claim 1, wherein predicting the at least one property of the first molecule is based at least in part upon prediction using (a) statistical modeling or machine learning and (b) chemical modeling.

3. The one or more non-transitory computer-readable media of claim 1, wherein predicting the at least one property of the first molecule comprises prediction using statistical modeling or machine learning, and then prediction using chemical modeling.

4. The one or more non-transitory computer-readable media of claim 1, storing instructions that, when executed, cause the first molecule to be obtained.

5. One or more non-transitory computer-readable media storing instructions for identifying a set of putative bioreachable molecules having one or more desired properties, wherein the instructions, when executed by one or more computing devices, cause at least one of the one or more computing devices to:
receive one or more queries indicating one or more desired properties;
determine data representing a set of putative bioreachable molecules based at least in part upon association of the set of putative bioreachable molecules with the one or more desired properties, wherein at least one of the one or more desired properties is based at least in part upon prediction using (a) statistical modeling or machine learning or (b) chemical modeling; and
return data representing the determined set.

6. The one or more non-transitory computer-readable media of claim 5, wherein at least one of the one or more desired properties is based at least in part upon prediction using statistical modeling or machine learning and at least one other of the desired properties is based at least in part upon prediction using chemical modeling.

7. The one or more non-transitory computer-readable media of claim 5, wherein the at least one of the one or more desired properties is based at least in part upon prediction using (a) statistical modeling or machine learning and (b) chemical modeling.

8. The one or more non-transitory computer-readable media of claim 5, wherein at least one of the one or more desired properties is initially based at least in part upon prediction using statistical modeling or machine learning, and the determined set comprises a reduced number of putative bioreachable molecules after later predicting at least one of the one or more desired properties using chemical modeling.

9. The one or more non-transitory computer-readable media of claim 5, wherein determining comprises determining data representing a set of putative bioreachable molecules based at least in part upon (a) association of the set of putative bioreachable molecules with the one or more desired properties and (b) a maximum distance between (i) one or more putative bioreachable molecules within the set and (ii) an ancestor putative bioreachable molecule satisfying a desired maximum distance.

10. The one or more non-transitory computer-readable media of claim 5 storing instructions that, when executed, cause the first molecule to be obtained.

11. One or more non-transitory computer-readable media storing instructions for predicting at least one property of a material related to a first molecule of one or more putative bioreachable molecules, wherein the instructions, when executed by one or more computing devices, cause at least one of the one or more computing devices to:
access a predictive model that employs (a) chemical modeling or (b) statistical modeling or machine learning;
predict at least one property of the material based at least in part upon the predictive model; and
return data representing the at least one property.

12. The one or more non-transitory computer-readable media of claim 11, wherein predicting the at least one property of the material is based at least in part upon prediction using (a) chemical modeling and (b) statistical modeling or machine learning.

13. The one or more non-transitory computer-readable media of claim 11, wherein predicting the at least one property of the material comprises prediction using statistical modeling or machine learning, and then prediction using chemical modeling.

14. The one or more non-transitory computer-readable media of claim 11, wherein the material comprises in its chemical structure at least the first molecule or at least one semi-synthetic molecule derived from the first molecule, or a combination thereof.

15. The one or more non-transitory computer-readable media of claim 11, storing instructions that when executed cause the first molecule to be obtained.

16. One or more non-transitory computer-readable media storing instructions for identifying one or more materials having one or more desired material properties, wherein the instructions, when executed by one or more computing devices, cause at least one of the one or more computing devices to:
receive one or more queries indicating one or more desired material properties;
determine data representing a set of materials based at least in part upon association of the set of materials with the one or more desired material properties, wherein the set of materials is related to one or more putative bioreachable molecules, and at least one of the one or more desired material properties is based at least in part upon prediction using (a) chemical modeling or (b) statistical modeling or machine learning; and
return data representing the determined set.

17. The one or more non-transitory computer-readable media of claim 16, wherein at least one of the one or more desired material properties is based at least in part upon prediction using chemical modeling and at least one other of the desired material properties is based at least in part upon prediction using statistical modeling or machine learning.

18. The one or more non-transitory computer-readable media of claim 16, wherein the at least one of the one or more desired material properties is based at least in part upon prediction using (a) chemical modeling and (b) statistical modeling or machine learning.

19. The one or more non-transitory computer-readable media of claim 16, wherein at least one of the one or more desired material properties is initially based at least in part upon prediction using statistical modeling or machine learning, and the determined set comprises a reduced number of materials after later predicting at least one of the one or more desired material properties using chemical modeling.

20. The one or more non-transitory computer-readable media of claim 16, wherein determining comprises determining data representing a set of materials based at least in part upon (a) association of the set of materials with the one or more desired material properties and (b) a maximum distance between (i) one or more putative bioreachable molecules related to the set and (ii) an ancestor putative bioreachable molecule satisfying a desired maximum distance.

21. The one or more non-transitory computer-readable media of claim 16, wherein the set of materials comprises one or more materials that each comprise in its chemical structure at least one of the one or more putative bioreachable molecules, or at least one semi-synthetic molecule related to at least one of the one or more putative bioreachable molecules, or a combination thereof.

22. The one or more non-transitory computer-readable media of claim 16 storing instructions that when executed cause at least one material of the set of materials or at least one of the one or more putative bioreachable molecules to be obtained.

23. One or more non-transitory computer-readable media storing instructions for identifying one or more putative bioreachable molecules related to one or more desired material properties, wherein the instructions, when executed by one or more computing devices, cause at least one of the one or more computing devices to:
receive one or more queries indicating one or more desired material properties; and
determine data representing a set of putative bioreachable molecules based at least in part upon association of the set of putative bioreachable molecules with the one or more desired material properties, wherein the set of putative bioreachable molecules is related to one or more materials, and at least one of the one or more desired material properties is based at least in part upon prediction using (a) chemical modeling or (b) statistical modeling or machine learning; and
return data representing the determined set.

24. The one or more non-transitory computer-readable media of claim 23, wherein at least one of the one or more desired material properties is based at least in part upon prediction using chemical modeling and at least one other of the desired material properties is based at least in part upon prediction using statistical modeling or machine learning.

25. The one or more non-transitory computer-readable media of claim 23, wherein the at least one of the one or more desired material properties is based at least in part upon prediction using (a) chemical modeling and (b) statistical modeling or machine learning.

26. The one or more non-transitory computer-readable media of claim 23, wherein at least one of the one or more desired material properties is initially based at least in part upon prediction using statistical modeling or machine learning, and the determined set comprises a reduced number of putative bioreachable molecules after later predicting at least one of the one or more desired material properties using chemical modeling.

27. The one or more non-transitory computer-readable media of claim 23, wherein determining comprises determining data representing a set of putative bioreachable molecules based at least in part upon (a) association of the set of putative bioreachable molecules with the one or more desired material properties and (b) a maximum distance between (i) one or more putative bioreachable molecules of the set and (ii) an ancestor putative bioreachable molecule satisfying a desired maximum distance.

28. The one or more non-transitory computer-readable media of claim 23, wherein the one or more related materials each includes within its chemical structure at least one putative bioreachable molecule of the set, at least one semi-synthetic molecule, or a combination thereof.

29. The one or more non-transitory computer-readable media of claim 23 storing instructions that when executed cause at least one of the one or more materials or at least one putative bioreachable molecule of the set to be obtained.

30. One or more non-transitory computer-readable media storing instructions for predicting properties of molecules derived from putative bioreachable molecules, wherein the instructions, when executed by one or more computing devices, cause at least one of the one or more computing devices to:
a. transform in silico a first putative bioreachable molecule of one or more putative bioreachable molecules to produce a second molecule in silico;
b. predict at least one property of the second molecule based at least in part upon (a) chemical modeling applied to the second molecule or (b) statistical modeling or machine learning; and
c. return data representing the at least one property.

31. The one or more non-transitory computer-readable media of claim 30, wherein predicting the at least one property of the second molecule is based at least in part upon prediction using (a) statistical modeling or machine learning and (b) chemical modeling.

32. The one or more non-transitory computer-readable media of claim 30, wherein predicting the at least one property of the second molecule comprises prediction using statistical modeling or machine learning, and then prediction using chemical modeling.

33. The one or more non-transitory computer-readable media of claim 30, wherein transforming includes chemically transforming.

34. The one or more non-transitory computer-readable media of claim 30, wherein transforming requires at most 2 reaction steps.

35. The one or more non-transitory computer-readable media of claim 30 storing instructions that when executed cause the second molecule to be obtained.

36. The one or more non-transitory computer-readable media of claim 1, wherein the one or more putative bioreachable molecules are determined by:
a. selecting reactions based at least in part upon whether the reactions are indicated as catalyzed by one or more corresponding catalysts that are themselves indicated as available to catalyze the reactions, wherein a reaction set comprises the selected reactions; and
b. in each processing step of one or more processing steps, processing, pursuant to the one or more reactions in the reaction set, data representing starting metabolites and metabolites generated in previous processing steps, to generate data representing the one or more putative bioreachable molecules.

37. The one or more non-transitory computer-readable media of claim 36, wherein selecting comprises selecting reactions that are indicated as catalyzed by one or more corresponding catalysts that are themselves indicated as able to be engineered into an organism or taken up from the growth medium in which an organism is grown.

38. The one or more non-transitory computer-readable media of claim 36, wherein selecting comprises selecting reactions that are indicated as catalyzed by one or more corresponding catalysts that are themselves indicated as corresponding to one or more amino acid sequences or one or more genetic sequences.

39. The one or more non-transitory computer-readable media of claim 36, wherein selecting comprises selecting reactions based at least in part upon whether the reactions are indicated in at least one database as catalyzed by one or more corresponding catalysts that are themselves indicated as available to catalyze the reactions.
